(19) United States
(12) Patent Application Publication
(10) Pub. No.: US 2021/0355545 A1
(43) Pub. Date: Nov. 18, 2021

(54) METHODS AND MATERIALS FOR ASSESSING AND TREATING CANCER

(71) Applicants: The Johns Hopkins University, Baltimore, MD (US); The Regents of the University of California, Oakland, CA (US)

(72) Inventors: Victor E. Velculescu, Dayton, MD (US); Robert B. Scharpf, Baltimore, MD (US); Eniko Papp, Baltimore, MD (US); Dorothy Hallberg, Baltimore, MD (US); Dennis Slamon, Oakland, CA (US); Gottfried Konecny, Oakland, CA (US)

(21) Appl. No.: 17/284,948

(22) PCT Filed: Oct. 15, 2019
(86) PCT No.: PCT/US2019/056299

§ 371 Date: Apr. 13, 2021

Related U.S. Application Data
(60) Provisional application No. 62/745,935, filed on Oct. 15, 2018.

(51) Int. Cl. C12Q 1/6886

(52) U.S. Cl. CPC C12Q 1/6886; C12Q 2600/156; C12Q 2600/154; C12Q 2600/106

(57) ABSTRACT

This document relates to methods and materials for assessing and/or treating mammals (e.g., humans) having cancer. For example, methods and materials for identifying a mammal as being likely to respond to a particular cancer treatment, and, optionally, for treating the mammal, are provided.

### CROSS-REFERENCE To RELATED APPLICATIONS

This application claims the benefit of U.S. Patent Application Ser. No. 62/745,935, filed on Oct. 15, 2018. The disclosure of the prior application is considered part of (and is incorporated by reference in) the disclosure of this application.

### STATEMENT REGARDING FEDERAL FUNDING

This invention was made with government support under CA121113, CA006973, and CA180950 awarded by National Institutes of Health. The government has certain rights in the invention.

### BACKGROUND

### 1. Technical Field

This document relates to methods and materials for assessing and/or treating mammals (e.g., humans) having cancer. For example, this document provides methods and materials for identifying a mammal as being likely to respond to a particular cancer treatment, and, optionally, the mammal can be treated.

### 2. Background Information

Ovarian cancer is the most common cause of death among gynecological cancers. Despite significant advances in therapies for other solid tumor malignancies, the overall survival of patients with late-stage ovarian cancer has remained dismal with few new options for treatment. The standard therapy involves debulking surgery followed by chemotherapy. Part of the reason for the lack of novel therapies for ovarian cancer has been an inadequate understanding of the underlying molecular characteristics of this disease, especially in the context of cancer cell models than can facilitate the development of various cancer treatments.

Recent studies have highlighted the genomic complexity and heterogeneity of ovarian cancer. These have included a catalog of sequence mutations, focal changes in DNA copy number, gene expression, and methylation alterations in high grade serous ovarian cancer (Network, 2011 Nature 474:609-615), as well as whole exome analyses of ovarian clear cell carcinoma and low grade serous carcinoma (Jones et al., 2012 J. Pathol. 226:413-420; and Jones et al., 2015 Science Translational Medicine 7:283ra53). Genome-wide sequence analyses of high grade serous ovarian cancer identified drivers associated with primary and acquired resistance to chemotherapy (Patch et al., 2015 Nature 521:489-494; Labidi-Galy et al., 2017 Nature communications 8:1093). More recently, a catalog of proteomic alterations in high grade serous TCGA samples has been integrated with structural alterations and correlated with clinical outcomes (Zhang et al., 2016 Cell 166:755-765). Hypothesis-generating pharmacogenomic studies involving cancer cell lines, some of which were ovarian, have revealed genetic- and expression-based alterations associated with resistance or sensitivity to a panel of drugs (Garnett et al., 2012 Nature 483:570-575; and Barretina et al., 2012 Nature 483:603-607). More recent cell line studies have evaluated high grade serous, clear cell and other cancers using targeted genomic and other molecular analyses (Domcke et al., 2013 Nature Communications 4:2126; Anglesio et al., 2013 PloS one 8:e72162; and Ince et al., 2015 Nature communications 6:7419). These initial efforts were extended to demonstrate the similarity of molecular alterations in cell lines to those in corresponding tissues, to develop approaches for incorporating multiple data types to model sensitivity, and to apply these models to larger drug panels (Iorio et al., 2016 Cell 166:740-754). Despite these advances, a comprehensive analysis of genome-wide structural alterations, including intra- and interchromosomal translocations and gene fusions, and integration of these data with whole-exome sequence, epigenetic and expression information are not available for many histological subtypes of ovarian cancer. Furthermore, the therapeutic response of these ovarian cancer subtypes to common targeted therapies is not well understood.

### SUMMARY

This document provides methods and materials for assessing and/or treating mammals (e.g., humans) having cancer. For example, a sample (e.g., a sample obtained from a mammal having or suspected of having a cancer) can be assessed for the presence or absence of one or more structural alterations. For example, this document provides methods and materials for identifying a mammal as being likely to respond to a particular cancer treatment, and, optionally, the mammal can be treated. In some cases, a mammal can be identified as having a cancer that is likely to respond to one or more poly(ADP-ribose) polymerase (PARP) inhibitors, based at least in part, on the mammal having one or more cancer cells having a MYC amplification (e.g., a focal MYC amplification), and, optionally, the mammal can be treated by administering one or more PARP inhibitors to the mammal. In some cases, a mammal can be identified as having a cancer that is likely to respond to one or more PARP inhibitors, based at least in part, on the mammal having one or more cancer cells having one or more genome-wide rearrangements, and, optionally, the mammal can be treated by administering one or more PARP inhibitors to the mammal. In some cases, a mammal can be identified as having a cancer that is likely to respond to one or more mitogen-activated protein kinase (MEK) inhibitors, based at least in part, on the mammal having one or more cancer cells having one or more modifications (e.g., one or more loss-of-function modifications) in SMAD3 and/or SMAD4, and, optionally, the mammal can be treated by administering one or more MEK inhibitors to the mammal. In some cases, a mammal can be identified as having a cancer that is likely to respond to one or more phosphatidylinositol 3-kinase (PI3K) inhibitors, based at least in part, on the mammal having one or more cancer cells having one or more modifications (e.g., one or more loss-of-function modifications) in PI3K CATALYTIC, ALPHA (PIK3CA) and/or protein phosphatase 2 scaffold subunit alpha (PPP2R1A), and, optionally, the mammal can be treated by administering one or more PI3K inhibitors to the mammal.

As demonstrated herein, a novel approach (e.g., Trellis) can be used for genomic analyses (e.g., detection of somatic sequence and structural changes) of tumors lacking matched normal samples. For example, genome-wide sequencing analyses of 45 ovarian cancer cell lines of varying subtypes was performed, Trellis was used for detection of somatic sequence and structural changes, and the detected somatic sequence and structural changes were integrated with epigenetic and expression alterations. Genetic modifications not previously implicated in ovarian cancer that are biologically and clinically relevant included amplification or overexpression of ASXL1 and H3F3B, deletion or underexpression of CDC73 and TGF beta receptor pathway members, and rearrangements of YAP1-MAML2 and IKZF2-ERBB4. Dose-response analyses to targeted therapies revealed novel molecular dependencies, including increased sensitivity of tumors with PIK3CA and PPP2R1A alterations to PI3K inhibitor GNE-493, MYC amplifications to PARP inhibitor BMN673, and SMAD3/4 alterations to MEK inhibitor MEK162. Also as demonstrated herein, genome-wide rearrangements provided an improved measure of sensitivity to PARP inhibition rather than the currently used homologous recombination deficiency (HRD) score.

The ability to identify genetic modifications not previously implicated in particular cancers provides clinicians with opportunities to detect cancers at earlier stages, to treat subjects more effectively, and/or to develop new therapeutics.

In some embodiments, provided herein are methods for assessing therapeutic benefit of a therapeutic regimen that includes a PARP inhibitor in a subject that include: detecting the presence of a MYC amplification in a tumor sample obtained from the subject, and identifying that the subject will have a predicted therapeutic benefit from the PARP inhibitor when the presence of the MYC amplification is detected in the tumor sample, wherein the therapeutic benefit for the subject is improved relative to the therapeutic benefit of the PARP inhibitor for a reference subject having a corresponding reference tumor sample that does not exhibit the MYC amplification. In some embodiments, provided herein are methods for assessing therapeutic benefit of a therapeutic regimen that includes a PARP inhibitor in a subject that include: detecting the presence of a plurality of genome-wide rearrangements in a tumor sample obtained from the subject, and identifying that the subject will have a predicted therapeutic benefit from the PARP inhibitor when the presence of the plurality of genome-wide rearrangements is detected in the tumor sample, wherein the therapeutic benefit for the subject is improved relative to the therapeutic benefit of the PARP inhibitor for a reference subject having a corresponding reference tumor sample that does not exhibit the plurality of genome-wide rearrangements. In some embodiments, provided herein are methods for assessing therapeutic benefit of a therapeutic regimen comprising a PARP inhibitor in a subject determined to have a MYC amplification in a tumor sample obtained from the subject that include: identifying that the subject will have a predicted therapeutic benefit from the PARP inhibitor when the presence of the MYC amplification is detected in the tumor sample, wherein the therapeutic benefit for the subject is improved relative to the therapeutic benefit of the PARP inhibitor for a reference subject having a corresponding reference tumor sample that does not exhibit the MYC amplification. In some embodiments, provided herein are methods for assessing therapeutic benefit of a therapeutic regimen comprising a PARP inhibitor in a subject determined to have a plurality of genome-wide rearrangements in a tumor sample obtained from the subject that include: identifying that the subject will have a predicted therapeutic benefit from the PARP inhibitor when the presence of the plurality of genome-wide rearrangements is detected in the tumor sample, wherein the therapeutic benefit for the subject is improved relative to the therapeutic benefit of the PARP inhibitor for a reference subject having a corresponding reference tumor sample that does not exhibit the plurality of genome-wide rearrangements. In some embodiments, the PARP inhibitor is one or more of talazoparib (BMN-673), olaparib (AZD-2281), rucaparib (PF-01367338), niraparib (MK-4827), veliparib (ABT-888), CEP 9722, E7016, BGB-290, iniparib (BSI 201), 3-aminobenzamide, and combinations thereof.

In some embodiments, provided herein are methods for assessing therapeutic benefit of a therapeutic regimen that includes a MEK inhibitor in a subject that include: detecting the presence of a SMAD3 or SMAD4 mutation in a tumor sample obtained from the subject, and identifying that the subject will have a predicted therapeutic benefit from the MEK inhibitor when the presence of the SMAD3 or SMAD4 mutation is detected in the tumor sample, wherein the therapeutic benefit for the subject is improved relative to the therapeutic benefit of the MEK inhibitor for a reference subject having a corresponding reference tumor sample that does not exhibit the SMAD3 or SMAD4 mutation. In some embodiments, provided herein are methods for assessing therapeutic benefit of a therapeutic regimen that includes a MEK inhibitor in a subject determined to have a SMAD3 or SMAD4 mutation in a tumor sample obtained from the subject that include: identifying that the subject will have a predicted therapeutic benefit from the MEK inhibitor identifying that the subject will have a when the presence of the SMAD3 or SMAD4 mutation is detected in the tumor sample, wherein the therapeutic benefit for the subject is improved relative to the therapeutic benefit of the MEK inhibitor for a reference subject having a corresponding reference tumor sample that does not exhibit the SMAD3 or SMAD4 mutation. In some embodiments, the MEK inhibitor is one or more of binimetinib (MEK162), trametinib (GSK1120212), cobimetinib (XL518), selumetinib, PD-325901, CI-1040, PD035901, TAK-733, and combinations thereof.

In some embodiments, provided herein are methods for assessing therapeutic benefit of a therapeutic regimen that includes a PI3K inhibitor in a subject that include: detecting the presence of a PIK3CA or PPP2R1A mutation in a tumor sample obtained from the subject, and identifying that the subject will have a predicted therapeutic benefit from the PI3K inhibitor identifying that the subject will have a when the presence of the PIK3CA or PPP2R1A mutation is detected in the tumor sample, wherein the therapeutic benefit for the subject is improved relative to the therapeutic benefit of the PI3K inhibitor for a reference subject having a corresponding reference tumor sample that does not exhibit the PIK3CA or PPP2R1A mutation. In some embodiments, provided herein are methods for assessing therapeutic benefit of a therapeutic regimen that includes a PI3K inhibitor in a subject determined to have a PIK3CA or PPP2R1A mutation in a tumor sample obtained from the subject that include: identifying that the subject will have a predicted therapeutic benefit from the PI3K inhibitor identifying that the subject will have a when the presence of the PIK3CA or PPP2R1A mutation is detected in the tumor sample, wherein the therapeutic benefit for the subject is improved relative to the therapeutic benefit of the PI3K inhibitor for a reference subject having a corresponding reference tumor sample that does not exhibit the PIK3CA or PPP2R1A mutation. In some embodiments, the PI3K inhibitor is one or more of GNE-493, wortmannin, demethoxyviridin, LY294002, hibiscone C, idelalisib, copanlisib, duvelisib, taselisib, perifosine, buparlisib, alpelisib (BYL719), umbralisib (TGR 1202), PX-866, dactolisib, CUDC-907, voxtalisib (SAR245409, XL765), ME-401, IPI-549, SF1126, RP6530, INK1117, pictilisib, XL147 (also known as SAR245408), palomid 529, GSK1059615, ZSTK474, PWT33597, IC87114, TG100-115, CAL263, RP6503, PI-103, GNE-477, AEZS-136, and combinations thereof.

In some embodiments of assessing therapeutic benefit of a therapeutic regimen in a subject, the tumor sample is an ovarian tumor sample. In some embodiments, the methods further include administering a therapeutic regimen to the subject. In some embodiments, the therapeutic regimen is one or more of: adoptive T cell therapy, radiation therapy, surgery, administration of a chemotherapeutic agent, administration of an immune checkpoint inhibitor, administration of a targeted therapy, administration of a kinase inhibitor, administration of a signal transduction inhibitor, administration of a bispecific antibody, administration of a monoclonal antibody, and combinations thereof.

In some embodiments, provided herein are methods of identifying a cancer-associated alteration in a sample obtained from a subject in the absence of a matched normal sample from the subject that include: (a) detection of germline changes, artifactual changes, or both, wherein the detected germline changes and detected artifactual changes are identified as not being a cancer-associated alteration; (b) detecting the presence of focal homozygous deletions, focal homozygous amplifications, or both, wherein the focal homozygous deletions and focal homozygous amplifications are distinguishable from larger structural changes; (c) associating one or more copy number regions; (d) detecting homozygous and hemizygous deletions; (e) detecting rearrangements using a stringent local re-alignment to detect and remove spurious paired read and split alignments; and (f) identifying in-frame rearrangements. In some embodiments, the step of detecting germline changes, artifactual changes, or both includes applying sequence and germline filters to flag regions prone to alignment artifacts, germline structural variations, or both. In some embodiments, the step of associating one or more copy number regions includes generating a plurality of amplicons and comparing paired sequences in the amplicons. In some embodiments, the step of comparing paired sequences in amplicons includes generating an undirected graph in which amplicons as nodes and in which edges between amplicons are generated by multiple paired sequencing reads aligned genomic locations associated with the amplicons. In some embodiments, the step of detecting homozygous and hemizygous deletions includes detecting copy number changes and rearrangements. In some embodiments, the identified in-frame rearrangements result in gene fusions.

In some embodiments, methods of identifying a cancer-associated alteration in a sample obtained from a subject in the absence of a matched normal sample from the subject indicates the presence of cancer in the subject. In some embodiments, methods of identifying a cancer-associated alteration in a sample obtained from a subject in the absence of a matched normal sample from the subject further include detecting methylation status of one or more genetic loci, which genetic loci are associated with the presence of cancer. In some embodiments, the methods further include administering a therapeutic regimen to the subject. In some embodiments, the therapeutic regimen is one or more of: adoptive T cell therapy, radiation therapy, surgery, administration of a chemotherapeutic agent, administration of an immune checkpoint inhibitor, administration of a targeted therapy, administration of a kinase inhibitor, administration of a signal transduction inhibitor, administration of a bispecific antibody, administration of a monoclonal antibody, and combinations thereof. In some embodiments, the sample is a tumor sample. In some embodiments, the sample is a liquid biopsy sample.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 shows and overview of genomic, epigenetic, and therapeutic analyses of ovarian cancer cell lines.

FIGS. 2A and 2B show a number of false positive somatic structural variant identifications in the lymphoblastoid cell lines. Assuming that nearly all of the rearrangements and copy number alterations in the lymphoblastoid cell lines are germline, the specificity of structural variant methods for identifying somatic structural variants were assessed by leave one out cross validation. FIG. 2A contains graphs showing the number of false positive somatic deletions and duplications/amplifications identified in each test sample stratified by size. FIG. 2B contains graphs showing the number of false positive somatic intra-chromosomal and inter-chromosomal rearrangements.

FIGS. 3A-3D show a Trellis approach for characterization of genomic structural alterations. FIG. 3A contains a circos plot displaying focal deletions (green), amplifications ( ), and intra- and inter-chromosomal rearrangements identified by rearranged read pairs and split reads. FIG. 3B contains a graph showing improperly paired established connections between distant amplicons, creating amplicon groups. Each amplicon group was visualized by a graph. The nodes of the graph are amplicons and the edges indicate multiple paired reads supporting the link. The size of the plotting symbols is proportional to the number of sites in which the amplicon was inserted and the triangle shape indicates an amplicon involving a known driver. For cell line FU-OV-1, there is only one amplicon group that involves 4 potential drivers (FGFR4, MYC, H3F3B, and CCNE1). FIG. 3C contains graphs showing the average maximum copy number of amplicon groups containing drivers was 18.3 compared to 7.8 in amplicon groups without any known drivers (top graph; t=3.3, 95% CI for mean difference: 4.0-17.0, p=0.003), and the mean number of amplicon links was 62 for amplicon groups containing drivers compared to 3.5 for amplicon groups without known drivers (bottom graph; t=5.3, p<0.001). FIG. 3D contains graphs showing segmented normalized coverage identified a homozygous deletion (top graph; shaded), and rearranged read pairs improved the precision of the deletion breakpoints. Lines connecting the read pairs indicate whether the positive or negative strand was sequenced (bottom graph; blue positive, green negative).

FIGS. 4A-4C show methylation of CpG sites in ovarian cancers and normal fallopian tissue. FIG. 4A shows the proportion of methylated CpG sites (mean β>0.3) in the lymphoblastoid cell lines, ovarian cell lines, TCGA ovarian cancers, and TCGA normal fallopian tissues. FIG. 4B shows 96 probes identified as being differentially methylated between normal TCGA fallopian tissue and 100 randomly selected (blue points, FIG. 4A) TCGA ovarian tumors. The lymphoblastoid and ovarian cancer cell lines were excluded from the probe selection procedure. As expected, this probe selection drives two major clusters separating TCGA fallopian tissue (left) from a large fraction of the TCGA ovarian tumors (right). Interestingly the lymphoblastoid cell lines were most correlated with normal fallopian tissue and the ovarian cell lines were most correlated with TCGA ovarian tumors, suggesting that the cell line effect does not dominate among probes that were differentially methylated in these tissues. Among probes that were methylated in TCGA ovarian and unmethylated in TCGA fallopian, the ovarian cell lines were predominantly methylated and have quantitatively higher β values. While copy number analyses suggested that the purity in the ovarian cell lines was ≈100%, the median tumor purity of TCGA ovarian tumors was 85% (interquartile range 78%-88%). FIG. 4C shows that genes CDKN2A and ESR1 exhibit bimodal gene expression explained by homozygous copy number deletions (blue points in x-axis margin) or methylation levels above 0.2. As the magnitude and heterogeneity of expression is gene-specific, a gene-specific threshold (dashed line) was used to determine under-expression.

FIG. 5 shows sequence, structural, and expression alterations in 10 clinically relevant pathways. Cell lines were grouped by tumor subtype (E=Endometrioid, Und=Undifferentiated, M=Mixed). For each pathway, genes were ordered by the frequency of a genomic alteration across 45 ovarian cell lines. For many of the pathways, mutual exclusivity of genomic alterations within the pathway is evident (e.g., cell cycle, TK receptors, TGFBR, BRCA, and WNT). The group indicated as Other contains genes that are clinically relevant for ovarian cancer but cannot be easily categorized by a single molecular process. Methylation and expression were not evaluated for the Large Gene group.

FIGS. 6A and 6B show sensitivity and resistance to pathway inhibitors. To identify genetic, epigenetic, and/or expression alterations influencing sensitivity to inhibitors of PARP (BMN673), PI3K (GNE-493), and MEK (MEK162) we used Bayesian model averaging. Candidate features for these models included genes with alterations in three or more ovarian tumors, as well as indicators for whether the square-root transformed number of rearrangements or square-root transformed HRD score was higher than the average of these statistics across all tumors. FIG. 6A shows features selected in fewer than half of the multi-variate models in the Monte Carlo simulations have a posterior probability of being non-zero ≤0.5 (vertical dashed line, left) and a posterior median of zero (right). FIG. 6B contains boxplots of inhibitor concentrations for features selected by Bayesian model averaging, as well as HRD, PARP1, and BRCA1/2 (left). The two cell lines with BRCA1/2 mutations are indicated by triangles in the PARP pathway. Right: The difference in mean logIC₅₀ concentrations by alteration status and the 90% highest posterior density (HPD) interval for the difference. For example, mutations in PIK3CA or PPP2R1A were associated with a −0.63 decrease in the average logIC₅₀, corresponding to a 48% increased sensitivity to the inhibitor GNE-493 (90% HPD: 5-72).

FIGS. 7A-7B show mutation signature analyses. FIG. 7A shows the frequency of mutations in each of the 96 possible trinucleotide contexts aggregated to the level of ovarian cancer subtype. The endometrial, mixed, undifferentiated, and unclassified tumors were collapsed into the Other category. FIG. 7B shows the contribution of each mutation signature to each ovarian cancer subtype. The serous cell lines have signatures 1A and 15, corresponding to aging and defective DNA mismatch repair.

FIG. 8 shows a re-alignment of putative lymphoblastoid inter-chromosomal translocations. DELLY identified 435 inter-chromosomal rearrangements that were private to lymphoblastoid cell line CGH10N. Of these, 53 (12%) were supported by one or more split reads and a consensus sequence in the tumor genome for the rearrangement was reported by DELLY. It was found that 13 (25%) of the consensus sequences had a GC composition less than 20%, indicating low sequence complexity (AT repeats). To further investigate, each consensus sequence was re-aligned to the hg19 reference genome using the local alignment algorithm BLAT. In each panel, a point corresponds to a single BLAT alignment. For several sequences, it was not possible to identify BLAT records at the two regions reported by DELLY (e.g, sequences 1-5). Among sequences that overlap both regions reported by DELLY, multiple high quality alignments at other locations in the genome were often found (e.g., sequences 7, 13, 14, and 18). Similarly, sequences with a very high BLAT alignment score to both DELLY regions suggests that the two regions have a similar sequence composition (e.g., sequence 9, 23, 43, and 44). With these considerations, only three of the 53 sequences (sequences 6, 15, and 27) have a split read BLAT alignment consistent with the rearrangement reported by DELLY and are less likely to be explained by alignment artifacts. Because all of these regions have five or more paired end alignments reported by DELLY, it was determined whether a similar number of discordantly read pairs could be identified from the ELAND alignments. Only 14 of the 53 regions had improperly paired reads supported by ELAND. Of these, half have fewer than five discordantly paired reads and two have more than 100 discordantly paired reads.

FIG. 9 shows structural variants in ovarian cancer cell lines. Circos plots (left) depict copy number alterations as well as intra- and inter-chromosomal rearrangements. Many ovarian cell lines have amplicons that can be genomically linked. For example, CGOV2T (cell line Caov-3) had multiple amplicons that were linked by rearranged read pairs. The linked amplicons were visualized by a graph. Amplicons comprising the nodes and edges indicate amplicons that were linked by 5 or more read pairs. The size of each node is proportional to the number of edges that connect to other amplicons. Triangles denote amplicons spanning potential drivers.

FIG. 10 shows whole genome sequencing analysis identified a YAP1-MAML2 fusion in ovarian cell line ES-2. The locations of YAP1 (blue rectangle, positive strand) and MAML2 (beige rectangle, negative strand) are indicated as rectangles on the q-arm ideogram of chr11. Rearranged read pairs and split reads indicate an inversion where the 3′ end of YAP1 is fused to the 5′ end of MAML2 such that MAML2 is now under control of the YAP1 promoter. The full transcripts with shading indicating the spliced regions (between exons 6 and 7 of YAP1 and exons 1 and 2 of MAML2), and grey regions indicating the parts of the complete transcript missing in the fused transcript. The amino acid sequence of YAP1 fused to MAML2 at the locations indicated by the dashed lines is the same as the amino acid sequence in the full protein, indicating that the fusion is in-frame. Note, the breakpoint in MAML2 amino acid sequence (aa 172) is the exact same breakpoint previously reported in MECT1-MAML2 and MAML2-MECT1 fusions. Finally, the fused protein with acidic and Q-rich domains of MAML2 (not drawn) remain intact.

FIGS. 11A-11B show whole genome sequencing analyses identified a fusion of IKZF2 and ERBB4 in ovarian cell line ES-2. FIG. 11A shows support by rearranged read pairs and split reads for the gene fusion. The fusion involved the promoter and first three exons of IKZF2 and exons 2-27 of ERBB4. FIG. 11B shows that three probes on the Agilent 44k microarray interrogate the first three exons of IKZF2 and two probes interrogate exons 2-27 of ERBB4. The average expression for probes hybridizing to these regions (y-axis) are similarly elevated in cell line ES-2 (black), suggesting that the fusion transcript is over-expressed. For cell lines without IKZF2-ERBB4 fusion (gray), ERBB4 is expressed at lower levels and the relationship between expression of ERBB4 and IKZF2 appears random.

FIGS. 12A-12B show copy number and rearrangement analyses identified a fusion of SHANK2-CCND1 that involves amplification of CCND1 FIG. 12A shows rearranged read pairs and split reads support an in-frame SHANK2-CCND1 fusion. FIG. 12B shows that CCND1 was amplified in cell line ES-2 and this amplification also participated in a fusion with SHANK2 (black). Expression of CCND1 in tumor ES-2 is high relative to its expression in other cell lines without this fusion.

FIG. 13 shows under-expression of genes with homozygous and/or hemizygous deletions. The probability that a gene was under-expressed was estimated by a two-component hierarchical mixture model implemented in the R package CNPBayes. The horizontal dashed line is the maximum observed expression value for which a gene was under-expressed with posterior probability 0.5 or greater. The strip labels indicate the gene expression probe and, if methylation was detected, the probe from the methylation platform. Triangles indicate methylated CpG sites (>0:2).

FIG. 14 shows gene amplifications were often over-expressed. The probability that a gene was over-expressed was estimated by a two-component hierarchical mixture model implemented in the R package CNPBayes. The horizontal dashed line is the minimum observed expression value for which a gene was over-expressed with posterior probability 0.5 or greater. The strip labels indicate the gene expression probe.

### DETAILED DESCRIPTION

This document provides methods and materials for identifying one or more structural alterations (e.g., cancer-specific structural alterations) in a sample. For example, a sample (e.g., a sample obtained from a mammal having, or suspected of having, a cancer) can be assessed for the presence or absence of one or more structural alterations. In some cases, this document provides methods and materials for using Trellis to detect the presence or absence of one or more structural alterations. In some cases, the methods and materials described herein can be used to detect the presence or absence of one or more structural alterations in a sample obtained from a mammal, where the presence of one or more structural alterations can be used to identify the mammal as having a disease (e.g., a cancer) associated with one or more structural alterations. For example, the methods and materials described herein can be used to detect the presence or absence of one or more structural alterations in a sample obtained from a mammal, where the presence of one or more structural alterations can be used to identify the mammal as having a disease (e.g., a cancer) associated with one or more structural alterations, and as being likely to respond to a particular cancer treatment.

This document also provides methods and materials for assessing and/or treating mammals (e.g., humans) having, or suspected of having, a cancer. For example, methods and materials described herein can be used for identifying a mammal as being likely to respond to a particular cancer treatment, based at least in part in the presence or absence of one or more structural alterations, and, optionally, the mammal can be treated. In some cases, a mammal can be identified as having a cancer that is likely to respond to one or PARP inhibitors, based at least in part, on the mammal having one or more cancer cells having a MYC amplification (e.g., a focal MYC amplification), and, optionally, the mammal can be treated by administering one or more PARP inhibitors to the mammal. In some cases, a mammal can be identified as having a cancer that is likely to respond to one or more PARP inhibitors, based at least in part, on the mammal having one or more cancer cells having one or more genome-wide rearrangements, and, optionally, can be treated by administering one or more PARP inhibitors to the mammal. In some cases, a mammal can be identified as having a cancer that is likely to respond to one or more MEK inhibitors, based at least in part, on the mammal having one or more cancer cells having one or more modifications (e.g., one or more loss-of-function modifications) in SMAD3 and/or SMAD4, and, optionally, the mammal can be treated by administering one or more MEK inhibitors to the mammal. In some cases, a mammal can be identified as having a cancer that is likely to respond to one or more PI3K inhibitors, based at least in part, on the mammal having one or more cancer cells having one or more modifications (e.g., one or more loss-of-function modifications) in PIK3CA and/or PPP2R1A, and, optionally, the mammal can be treated by administering one or more PI3K inhibitors to the mammal.

Any type of mammal can be assessed and/or treated as described herein. A mammal can be a mammal having, or suspected of having, a cancer. A mammal can be a mammal suspected of having cancer. Examples of mammals that can be assessed and/or treated as described herein include, without limitation, humans, non-human primates (e.g., monkeys), dogs, cats, horses, cows, pigs, sheep, mice, and rats. In some cases, a mammal can be a human. For example, a human can be assessed for the presence or absence of one or more structural alterations as described herein and, based, at least in part on presence of one or more structural alterations described herein, can be identified as being likely to respond to a particular cancer treatment and, optionally, the mammal can be treated with one or more cancer particular treatments as described herein. For example, a human can be identified as being likely to respond to a particular cancer treatment based, at least in part on presence of one or more structural alterations described herein, and, optionally, the mammal can be treated with one or more cancer particular treatments as described herein.

Any appropriate sample from a mammal can be assessed as described herein (e.g., assessed for the presence of one or more structural alterations). For example, a sample can be obtained from a mammal (e.g., a mammal having, or suspected of having, a cancer), and can be assessed as described herein (e.g., assessed for the presence or absence of one or more structural alterations). In some cases, a sample can include one or more cancer cells. In some cases, a sample can be fluid sample. In some cases, a sample can be a tissue sample. In some cases, a sample can include DNA (e.g., genomic DNA). In some cases, a sample can include cell-free DNA (e.g., circulating tumor DNA (ctDNA)). A sample can be a fresh sample or a fixed sample. Examples of samples that can be assessed for one or more structural alterations (e.g., cancer-specific structural alterations) as described herein include, without limitation, ovarian tissue, pap smears, skin tissue, brain tissue, liver tissue, tumor tissue, spleen tissue, kidney tissue, heart tissue, lung tissue, blood (e.g., whole blood, serum, or plasma), amnion, tissue, urine, cerebrospinal fluid, synovial fluid, saliva, sputum, broncho-alveolar lavage, bile, lymphatic fluid, cyst fluid, stool, and ascites. For example, an ovarian tissue sample can be assessed for the presence or absence of one or more structural alterations (e.g., cancer-specific structural alterations) as described herein.

In some cases, a sample can be processed (e.g., to isolate and/or purify DNA and/or peptides from the sample). In some cases, a processed sample can be an embedded sample (e.g., a paraffin-embedded sample). For example, DNA isolation and/or purification can include cell lysis (e.g., using detergents and/or surfactants), protein removal (e.g., using a protease), and/or RNA removal (e.g., using an RNase). As another example, peptide isolation and/or purification can include cell lysis (e.g., using detergents and/or surfactants), DNA removal (e.g., using a DNase), and/or RNA removal (e.g., using an RNase).

Methods and materials for identifying one or more structural alterations (e.g., cancer-specific structural alterations) can include assessing a genome (e.g., a genome of a mammal) for the presence or absence of one or more structural alterations (e.g., cancer-specific structural alterations). In some cases, methods and materials for identifying one or more structural alterations as described herein also can be referred to as Trellis. The presence or absence of one or more structural alterations in the genome of a mammal can, for example, be determined using whole-genome sequence data (e.g., to characterize structural alterations such as amplifications and rearrangements). In some cases, one or more structural alterations in a genome (e.g., a genome of a mammal) can be identified in a sample obtained from a mammal (e.g., a mammal having, or suspected of having, a cancer). In some cases, methods and materials for identifying one or more structural alterations in a genome (e.g., a genome of a mammal) do not include a normal sample (e.g., a sample from a healthy mammal such as a mammal that does not have cancer). For example, when a sample is obtained from a mammal having a cancer, methods and materials described herein do not include a matched normal sample from the mammal (e.g., a sample including one or more healthy cells from the same mammal from which a sample including one or more cancer cells was obtained).

In some cases, methods and materials described herein can be used for identifying structural alterations that are linked (e.g., genomically linked). For example, methods and materials described herein can be used for identifying an amplification that includes both a copy number change and a rearrangement.

In some cases, methods and materials described herein can be used for identifying one or more structural alterations in a genome can include detecting cancer-specific structural alterations (e.g., through removal of germline and artifactual changes), distinguishing focal deletions and amplifications from larger structural changes, connecting apparently disparate copy number regions (e.g., using paired sequences in the same amplicons), detecting deletions (e.g., through copy number and rearrangement data), detecting rearrangements (e.g., using a stringent local re-alignment to detect and remove spurious paired read and split alignments), and identifying rearrangements that result in gene fusions (e.g., in-frame rearrangements). In some cases, identifying one or more structural alterations in a genome can be as described in Example 1.

In some cases, methods and materials for identifying one or more structural alterations as described herein can include using one or more germline filters and/or one or more sequence filters. A germline filter and/or a sequence filter can include a pool of one or more (e.g., one, two, three, four, five, six, seven, eight, nine, ten, eleven, twelve, or more) immortalized cell lines (e.g., lymphoblastoid cell lines) and one or more (e.g., one, two, three, four, five, six, seven, eight, nine, ten, eleven, twelve, or more) normal cells (e.g., cells from a sample obtained from a healthy mammal such as a mammal that does not have cancer). Examples of immortalized cells lines that can be used in a germline filter described herein include, without limitation, lymphoblastoid cell lines. An example of normal cells that can be used in a germline filter described herein include, without limitation, normal ovarian cells. For example, a pool of about 10 lymphoblastoid cell lines and cells from about 8 normal ovarian samples can be used to generate a germline filter and/or a sequence filter. In some cases, a germline filter and/or a sequence filter can be as described in Example 1.

A germline filter and/or a sequence filter can include any appropriate length of a genome. In some cases, a germline filter and/or a sequence filter can include from about 200 megabases (Mb) to about 500 Mb of a genome. For example, a germline filter and/or a sequence filter can include about 326.4 Mb of a genome. In some cases, the length of a germline filter and/or a sequence filter can be divided into intervals (bins). A length of a germline filter and/or a sequence filter can include any appropriate number of bins. In some cases, the length of a germline filter and/or a sequence filter can be divided into non-overlapping bins. A bin can be any appropriate size. In some cases, a bin can be from about 0.5 kilobases (kb) to about 5 kb. For example, a bin can be about 1 kb. A bin can have any appropriate mappability. For example, a bin can have a mappability of from about 0.25 to about 2. In some cases, a bin can have a mappability of less than about 0.75. A bin can have any appropriate GC percentage. For example, a bin can have a GC percentage of from about 5% to about 20%. In some cases, a bin can have a GC percentage of less than about 10%.

A germline filter and/or a sequence filter can be used to filter a reference genome to obtain a filtered reference genome. A reference genome can be any appropriate genome. In some cases, a reference genome can be as described elsewhere (see, e.g., the Genome Reference Consortium, the European Bioinformatics Institute, the National Center for Biotechnology Information, the Sanger Institute, and McDonnell Genome Institute). In some cases, a reference genome can be a human reference genome. Examples of reference genomes include, without limitation, hg38, hg19, hg18, hg17, and hg16. For example, a sequence filter can used to filter a hg19 reference genome. In some cases, using a germline filter and/or a sequence filter to filter a reference genome can identify regions of the genome that are prone to alignment artifacts and/or germline structural variation. In some cases, a sequence filter (e.g., a sequence filter for a hg19 reference genome) can be masked.

In some cases, a GC-adjusted and/or log2-transformed count of aligned reads for each bin of a read depth of a filtered reference genome can be computed. For example, a read depth of a filtered reference genome can be normalized for the remaining bins. In some cases, a read depth of a filtered reference genome can include from about 1 million to about 4 million bins. For example, a read depth of a filtered reference genome can include about 2,680,222 bins. In some cases, normalizing a read depth of a filtered reference genome can include GC-normalization. For example, GC-normalization can include using a loess smoother with span ⅓ fitted to a scatterplot of the bin-level GC and log2 count to obtain GC-adjusted log2 ratios (the residuals from the loess correction). For example, when the GC-adjusted log2 ratios are denoted by R, the mean R for a genomic region is \dot{R̅}, and the median absolute deviation of the autosomal Rs is S. In some cases, when a bin had a high or low number of aligned reads in multiple controls, the bin i was defined in normal control j as an outlier if |Ri|>(3×Sj). In some cases, somatic copy number alterations can be identified by segmenting the Rs (e.g., using circular binary segmentation). In some cases, copy number altered in the lymphoblastoid cell lines and/or segments that span difficult regions (e.g., segments having |R|>1) can be excluded.

In some cases, methods and materials described herein can be used for identifying one or more deletions (e.g., somatic deletions). A deletion can be a homozygous deletion. A deletion can be a hemizygous deletion. A deletion can be any appropriate size. For example a deletion can be from about 2 kb to about 3 Mb (e.g., from about 2 kb to about 2.5 Mb, from about 2 kb to about 2 Mb, from about 2 kb to about 1.5 Mb, from about 2 kb to about 1 Mb, from about 2 kb to about 0.5 Mb, from about 2.5 kb to about 3 Mb, from about 3 kb to about 3 Mb, from about 3.5 kb to about 3 Mb, from about 4 kb to about 3 Mb, from about 5 kb to about 3 Mb, from about 6 kb to about 3 Mb, from about 7 kb to about 3 Mb, or from about 8 kb to about 3 Mb). In some cases, a deletion that includes greater than about 75% (e.g., about 75%, about 80%, about 85%, about 90%, about 95%, about 98%, or greater) can be excluded. For example, a deletion greater than about 2 kb can be identified using the formula \dot{R̅}<−3. For example, a deletion less than about 3 Mb can be identified using the formula \dot{R̅}ϵ (−3; −0:75). In some cases, each deletion can be assessed for improperly paired reads (e.g., reads aligned within 5 kb of the segmentation boundaries). In cases where five or more read pairs are improperly paired, the distribution of the improper read pair alignments can be used to further resolve the genomic coordinates of the deletion boundaries. In some cases, resolution of deletion breakpoints can depends on the intra-mate distance of the improperly paired reads. For example, the intra-mate distance can be from about 100 bp to about 300 bp (e.g., about 262 bp). In some cases, deletion breakpoints can be less than about 100 bp. In some cases, a deletion can be confirmed (e.g., by visual inspection). In some cases, identifying one or more deletions can be as described in Example 1.

In some cases, methods and materials described herein can be used for identifying one or more amplifications (e.g., somatic amplifications). In some cases, methods and materials for identifying one or more amplifications also can determine whether or not two or more amplicons are linked. In some cases, amplifications can be identified using the formula R>1:46 and/or or a 2.75-fold increase from the mean ploidy of the cell line, and between 2 kb and 3 Mb in length. In some cases, properly paired reads can be used to link seed amplicons to adjacent low-copy duplications. For example, segments with R>0:81 or fold-change of 1.75 can be used to link seed amplicons to adjacent low-copy duplications. In some cases, identifying one or more amplifications can be as described in Example 1.

In some cases, methods and materials described herein can be used for identifying rearrangements (e.g., somatic rearrangements). A rearrangement can be a copy-neutral rearrangement. In some cases, rearrangements identified in one or more controls samples can be excluded. In some cases, a rearrangement can include one or more improperly paired reads (e.g., reads aligned within 5 kb of the segmentation boundaries). In cases where five or more read pairs are improperly paired, the distribution of the improper read pair alignments can be used to further resolve the genomic coordinates of the rearrangement boundaries. In some cases, a rearrangement can include one or more split reads. For example, a split read alignment can be identified by extracting all read pairs for which only one read in the pair was aligned within 5 kb of the candidate rearrangement. For all such read pairs, the unmapped mate can be re-aligned using BLAT (see, e.g., Kent, 2002 Genome Res 12:656-664). As used herein, a split read can include any BLAT alignment where the realigned read aligned to both ends of the candidate sequence junction with a combined score of the two alignments ≥90% constituted a split read. In some cases, identifying rearrangements can be as described in Example 1.

In some cases, methods and materials described herein can be used for identifying one or more gene fusions (e.g., in-frame gene fusions). A gene fusion can include a coding sequence of the genome. A gene fusion can include a promoter sequence (e.g., a sequence within 5 kb of a transcription start site). In some cases, two orientations of a fusion gene can be evaluated. For example, for each orientation the full amino acid sequence of both the 5′ and 3′ transcripts can be extracted as well as the candidate amino acid sequence that would be encoded by the fusion gene. In some cases, a fusion gene can be an in-frame fusion gene (e.g., a fusion gene that encodes a fusion polypeptide). In some cases, identifying one or more gene fusions can be as described in Example 1.

In some cases, methods and materials described herein can be used for identifying nucleic acid methylation. For example, processed (e.g., pre-processed) and normalized raw DAT files from the Infinimum MethylationEPIC array can be assessed for genome-wide methylation using the funnorm function in the R package minfi (see, e.g., Aryee et al. 2014 Bioinformatics 30:1363-1369). In some cases, one or more (e.g., probes on chromosomes X or Y, probes with detection p-value greater than 0.5, and/or probes overlapping a SNP with dbSNP minor allele frequency greater than 10%) can be excluded. For example, methylation can be assessed using Infinium HumanMethylation27 BeadChip array (27,578 probes). In some cases, the number of probes in common between the HumanMethylation27 platforms and the MethylationEPIC platform can be from about 10,000 to about 30,000. For example, the number of probes in common between the HumanMethylation27 platforms and the MethylationEPIC platform can be about 18,016. On a common set of probes, overall methylation can be quantified as the fraction of CpG sites with β>0:3, and differentially methylated CpG sites can be identified as hyper-methylated (average β>0:4) or unmethylated (average β<0:2). In addition, probes were also selected that were hypo-methylated in TCGA ovarian cancer (average β<0:1) and hyper-methylated in normal fallopian (average β>0:3). In some cases, identifying methylation can be as described in Example 1.

The methods and materials described herein can be used to identify any appropriate structural alterations. In some cases, a structural alteration can be a cancer-specific structural alteration. For example, a cancer-specific structural alteration can affect one or more driver genes. A structural alteration can be a genomic alteration. A structural alteration can be an epigenomic alteration. A structural alteration can be a transcriptomic alteration. A structural alteration can be a proteomic alteration. A structural alteration can be a metabolomic alteration. A structural alteration can be a carbohydrate alteration. Examples of structural alterations can include, without limitation, modifications, deletions, amplifications, rearrangements, epigenetic alterations, and post-translational modification alterations. In some cases, the presence or absence of one or more structural alterations a cancer cell within a mammal having, or suspected of having, a cancer can be used to identify the mammal as being likely to respond to a particular cancer treatment.

In some cases, a structural alteration can result in elevated levels (e.g., increased expression) of one or more polypeptides (e.g., one or more polypeptides encoded by a nucleic acid sequence having a structural alteration). The term “elevated level” as used herein with respect to a level of a polypeptide refers to any level that is greater than a reference level of the polypeptide, respectively. The term “reference level” as used herein with respect to one or more polypeptides refers to the level of a polypeptide typically observed in a sample (e.g., a control sample) from one or more mammals (e.g., humans) without cancer. Control samples can include, without limitation, matched normal samples from the same mammal from which a sample was obtained, samples from normal mammals (e.g., healthy mammals such as mammals that do not have cancer), and cell lines (e.g., non-tumor forming cells lines). In some cases, for example, when using a Trellis method as described herein, a control sample is not a matched normal sample. In some cases, an increased level of a polypeptide can be a level that is at least 2-fold (e.g., at least 3-fold, at least 4-fold, at least 5-fold, at least 6-fold, at least 7-fold, at least 8-fold, at least 9-fold, or at least 10-fold) greater than a reference level of the polypeptide. In some cases, when control samples have undetectable levels of a polypeptide, an elevated level can be a detectable level of the polypeptide. It will be appreciated that levels from comparable samples are used when determining whether or not a particular polypeptide is present at an elevated level.

In some cases, a structural alteration can result in decreased levels (e.g., decreased expression) of one or more polypeptides (e.g., one or more polypeptides encoded by a nucleic acid sequence having a structural alteration). The term “decreased levels” as used herein with respect to a level of a polypeptide refers to any level that is less than a reference level of the polypeptide, respectively. The term “reference level” as used herein with respect to one or more polypeptides refers to the level of a polypeptide typically observed in a sample (e.g., a control sample) from one or more mammals (e.g., humans) without cancer. Control samples can include, without limitation, matched normal samples from the same mammal from which a sample was obtained, samples from normal mammals (e.g., healthy mammals such as mammals that do not have cancer), and cell lines (e.g., non-tumor forming cells lines). In some cases, for example, when using a Trellis method as described herein, a control sample is not a matched normal sample. In some cases, a decreased level of a polypeptide can be a level that is at least 2-fold (e.g., at least 3-fold, at least 4-fold, at least 5-fold, at least 6-fold, at least 7-fold, at least 8-fold, at least 9-fold, or at least 10-fold) less than a reference level of the polypeptide. In some cases, when control samples have detectable levels of a polypeptide, a decreased level can be an undetectable level of the polypeptide. It will be appreciated that levels from comparable samples are used when determining whether or not a particular polypeptide is present at a decreased level.

In some cases, a structural alteration can be an amplification of a nucleic acid sequence (e.g., a coding sequence such as a gene amplification). An amplification can be a cancer-specific amplification. An amplification can result in a copy number change of a coding sequence (e.g., a gene). In some cases, a gene amplification can result in increased expression (e.g., increased levels) of a polypeptide encoded by the amplified gene. For example, a cancer cell within a mammal having, or suspected of having, a cancer can include one or more cancer-specific gene amplifications. An amplification can include amplification of any appropriate coding sequence (e.g., any appropriate gene). Examples of nucleic acid sequences that can be amplified in a cancer-specific amplification include, without limitation, a MYC nucleic acid sequence, a ASXL1 nucleic acid sequence, a H3F3B nucleic acid sequence, a ERBB2 nucleic acid sequence, a CCND1 nucleic acid sequence, a CCNE1 nucleic acid sequence, a FGFR4 nucleic acid sequence, a KRAS nucleic acid sequence, a NOTCH4 nucleic acid sequence, a RAD51C nucleic acid sequence, and a RNF43 nucleic acid sequence. In some cases, a coding sequence that can be amplified in a cancer-specific amplification can be as set forth in Table 7. For example, a cancer-specific amplification can be a MYC amplification (e.g., a focal MYC amplification).

In some cases, a structural alteration can be a rearrangement (e.g., a genome-wide rearrangement). A rearrangement can be a cancer-specific rearrangement. A rearrangement can be any appropriate type of rearrangement (e.g., deletions, duplications, inversions, and translocations). A rearrangement can be an intra-chromosomal rearrangement or inter-chromosomal rearrangement. When a rearrangement is an intra-chromosomal rearrangement, the rearrangement can include any appropriate chromosome. An intra-chromosomal rearrangement can include any chromosome pair (e.g., chromosome 1, chromosome 2, chromosome 3, chromosome 4, chromosome 5, chromosome 6, chromosome 7, chromosome 8, chromosome 9, chromosome 10, chromosome 11, chromosome 12, chromosome 13, chromosome 14, chromosome 15, chromosome 16, chromosome 17, chromosome 18, chromosome 19, chromosome 20, chromosome 21, chromosome 22, and/or one of the sex chromosomes (e.g., an X chromosome or a Y chromosome). When a rearrangement is an inter-chromosomal rearrangement, the rearrangement can include any appropriate type of nucleic acid sequence (e.g., a coding sequence such as a gene, a regulatory element such as a promoter and/or enhancer, or a splice site sequence). In some cases, a rearrangement can include a coding sequence (e.g., a gene). In some cases, a rearrangement can include a regulatory sequence (e.g., a promoter and/or enhancer). Examples of nucleic acid sequences that can be rearranged in a cancer-specific rearrangement include, without limitation, a MYC nucleic acid sequence, a YAP1 nucleic acid sequence, a MAML2 nucleic acid sequence, a IKZF2 nucleic acid sequence, a ERBB4 nucleic acid sequence, a CCND1 nucleic acid sequence, a SHANK2 nucleic acid sequence, a CCND1I nucleic acid sequence, a NF1 nucleic acid sequence, a TSC2 nucleic acid sequence, a FBXW7 nucleic acid sequence, a MLST8 nucleic acid sequence, and a FAM160A1 nucleic acid sequence. In some cases, a cancer-specific rearrangement can be as set forth in Table S9.

In some cases, a rearrangement can result in one or more fusion genes (e.g., a fusion gene encoding a fusion polypeptide). For example, a fusion gene can include a promoter that drives expression of a coding sequence (e.g., a first coding sequence) fused to a coding sequence of a different (e.g., a second) coding sequence. Examples of fusion genes that can result from a cancer-specific rearrangement include, without limitation, YAP1-MAML2, IKZF2-ERBB4, SHANK2-CCND1, NF1-MY01D,MLST8-TSC2, and FBXW7-FAM160A1. For example, a cancer-specific fusion gene can be a YAP1-MAML2. In some cases, a cancer-specific fusion gene can be as set forth in Table 10. For example, a cancer-specific fusion gene can be a IKZF2-ERBB4.

In some cases, a structural alteration can be a modification (e.g., a nucleic acid sequence modification). A modification can be a cancer-specific modification. A modification can be a homozygous modification. A modification can be a hemizygous modification. In some cases, a modification can be an activating modification. For example, an activating modification can include one or more modifications (e.g., insertions, substitutions, deletions, indels, and truncations) to a regulatory sequence (e.g., a promoter and/or enhancer) such that the regulatory sequence encodes an elevated level of a polypeptide. For example, an activating modification can include one or more modifications (e.g., insertions, substitutions, deletions, indels, and truncations) to a coding sequence (e.g., a gene) such that the coding sequence encodes a polypeptide having increased activity (e.g., constitutive activity). In some cases, a modification can be an inactivating modification. For example, an inactivating modification can include one or more modifications (e.g., insertions, substitutions, deletions, indels, and truncations) to a coding sequence (e.g., a gene) such that the coding sequence does not encode any polypeptide. For example, an inactivating modification can include one or more modifications (e.g., insertions, substitutions, deletions, indels, and truncations) to a coding sequence (e.g., a gene) such that the coding sequence encodes a non-functional polypeptide. In some cases, a modification can include modification of any appropriate regulatory element (e.g., a promoter and/or enhancer). In some cases, a modification can include modification of any appropriate coding sequence (e.g., a gene). A coding sequence can encode a cell cycle regulator. A coding sequence can encode a tyrosine kinase receptor. A coding sequence can encode a neurofibromin. A coding sequence can encode a transcriptional regulator. A coding sequence can encode a polycomb-group repressor. A coding sequence can encode a serine/threonine kinase. A coding sequence can encode a TGF beta pathway members. A coding sequence can encode a hormone receptor such as an estrogen receptor. A coding sequence can encode a cell cycle kinase. A coding sequence can encode a notch receptor. A coding sequence can encode a cohesin member. A coding sequence can encode an epigenetic regulator. Examples of nucleic acid sequences that can be modified in a cancer-specific modification include, without limitation, a PPP2R1A nucleic acid sequence, a PIK3CA nucleic acid sequence, a CDC73 nucleic acid sequence, a ERBB4 nucleic acid sequence, a EZH2 nucleic acid sequence, a MLH1 nucleic acid sequence, a TGFBR2 nucleic acid sequence, a SMAD3 nucleic acid sequence, a SMAD4 nucleic acid sequence, a ESR1 nucleic acid sequence, a CDK6 nucleic acid sequence, a NOTCH1 nucleic acid sequence, a STAG2 nucleic acid sequence, a ATRX nucleic acid sequence, a CDKN2A nucleic acid sequence, a CDKN2B nucleic acid sequence, a NF1 nucleic acid sequence, a NF2 nucleic acid sequence, a EZH2 nucleic acid sequence, a STK11 nucleic acid sequence, a TP53 nucleic acid sequence, a ARID1A nucleic acid sequence, a KRAS nucleic acid sequence, a APC nucleic acid sequence, and a CREBBP nucleic acid sequence. In some cases, a cancer-specific modification can be as set forth in Table S8. For example, a cancer-specific modification can be a modification in SMAD3 and/or SMAD4.

When assessing and/or treating a mammal having, or suspected of having, a cancer as described herein, the cancer can be any type of cancer. A cancer can be a primary cancer or a metastatic cancer. A cancer can be a hormone receptor positive cancer or a hormone receptor negative cancer. In some cases, a cancer can include one or more solid tumors. In some cases, a cancer can be a cancer in remission. In some cases, a cancer can include quiescent (e.g., dormant or non-dividing) cancer cells. In some cases, a cancer can be cancer that has escaped chemotherapy and/or has been non-responsive to chemotherapy. Examples of cancers that can be assessed and/or treated as described herein include, without limitation, ovarian cancers, breast cancers, pancreatic cancers, prostate cancers, lung cancer (e.g., small cell lung carcinoma or non-small cell lung carcinoma), papillary thyroid cancer, medullary thyroid cancer, differentiated thyroid cancer, recurrent thyroid cancer, refractory differentiated thyroid cancer, lung adenocarcinoma, bronchioles lung cell carcinoma, multiple endocrine neoplasia type 2A or 2B (MEN2A or MEN2B, respectively), pheochromocytoma, parathyroid hyperplasia, colorectal cancer (e.g., metastatic colorectal cancer), papillary renal cell carcinoma, ganglioneuromatosis of the gastroenteric mucosa, inflammatory myofibroblastic tumor, cervical cancer, acute lymphoblastic leukemia (ALL), acute myeloid leukemia (AML), cancer in adolescents, adrenal cancer, adrenocortical carcinoma, anal cancer, appendix cancer, astrocytoma, atypical teratoid/rhabdoid tumor, basal cell carcinoma, bile duct cancer, bladder cancer, bone cancer, brain stem glioma, brain tumor, bronchial tumor, Burkitt lymphoma, carcinoid tumor, unknown primary carcinoma, cardiac tumors, cervical cancer, childhood cancers, chordoma, chronic lymphocytic leukemia (CLL), chronic myelogenous leukemia (CML), chronic myeloproliferative neoplasms, colon cancer, colorectal cancer, craniopharyngioma, cutaneous T-cell lymphoma, bile duct cancer, ductal carcinoma in situ, embryonal tumors, endometrial cancer, ependymoma, esophageal cancer, esthesioneuroblastoma, Ewing sarcoma, extracranial germ cell tumor, extragonadal germ cell tumor, extrahepatic bile duct cancer, eye cancer, fallopian tube cancer, fibrous histiocytoma of bone, gallbladder cancer, gastric cancer, gastrointestinal carcinoid tumor, gastrointestinal stromal tumors (GIST), germ cell tumor, gestational trophoblastic disease, glioma, hairy cell tumor, hairy cell leukemia, head and neck cancer, heart cancer, hepatocellular cancer, histiocytosis, Hodgkin's lymphoma, hypopharyngeal cancer, intraocular melanoma, islet cell tumors, pancreatic neuroendocrine tumors, Kaposi sarcoma, kidney cancer, Langerhans cell histiocytosis, laryngeal cancer, leukemia, lip and oral cavity cancer, liver cancer, lymphoma, macroglobulinemia, malignant fibrous histiocytoma of bone, osteocarcinoma, melanoma, Merkel cell carcinoma, mesothelioma, metastatic squamous neck cancer, midline tract carcinoma, mouth cancer, multiple endocrine neoplasia syndromes, multiple myeloma, mycosis fungoides, myelodysplastic syndromes, myelodysplastic/myeloproliferative neoplasms, myelogenous leukemia, myeloid leukemia, multiple myeloma, myeloproliferative neoplasms, nasal cavity and paranasal sinus cancer, nasopharyngeal cancer, neuroblastoma, non-Hodgkin's lymphoma, oral cancer, oral cavity cancer, lip cancer, oropharyngeal cancer, osteosarcoma, hepatobiliary cancer, upper urinary tract cancer, papillomatosis, paraganglioma, paranasal sinus and nasal cavity cancer, parathyroid cancer, penile cancer, pharyngeal cancer, pheochromosytoma, pituitary cancer, plasma cell neoplasm, pleuropulmonary blastoma, primary central nervous system lymphoma, primary peritoneal cancer, rectal cancer, renal cell cancer, retinoblastoma, rhabdomyosarcoma, salivary gland cancer, sarcoma, Sezary syndrome, skin cancer, small intestine cancer, soft tissue sarcoma, squamous cell carcinoma, squamous neck cancer, stomach cancer, T-cell lymphoma, testicular cancer, throat cancer, thymoma and thymic carcinoma, thyroid cancer, transitional cell cancer of the renal pelvis and ureter, unknown primary carcinoma, urethral cancer, uterine cancer, uterine sarcoma, vaginal cancer, vulvar cancer, Waldenstrom and Macroglobulinemia. In some cases, a mammal (e.g., a human) having ovarian cancer can be assessed and/or treated as described herein. For example, a human having ovarian cancer can be assessed for the presence or absence of one or more structural alterations as described herein and, based, at least in part, on the presence of one or more structural alterations described herein, can be identified as being likely to respond to a particular cancer treatment and, optionally, the mammal can be treated with one or more cancer particular treatments as described herein. For example, a human having ovarian cancer can be identified as being likely to respond to a particular cancer treatment based, at least in part, on the presence of one or more structural alterations described herein, and, optionally, the mammal can be treated with one or more cancer particular treatments as described herein.

In some cases, a mammal can be identified as having a cancer. Any appropriate method can be used to identify a mammal as having a cancer. As non-limiting examples, imaging techniques, biopsy techniques, and/or liquid biopsy techniques can be used to identify mammals (e.g., humans) having cancer.

In some cases, a mammal having, or suspected of having, a cancer can be assessed to determine whether or not a cancer will or is likely to respond to a particular cancer treatment. For example, a sample obtained from the mammal can be assessed the presence or absence of one or more structural alterations (e.g., cancer-specific structural alterations), and the presence or absence of one or more structural alterations (e.g., cancer-specific structural alterations) can be used to determine whether or not the mammal will or is likely to respond to a particular cancer treatment.

In some cases, the presence of absence of one or more amplifications (e.g., amplifications of a coding sequence such as a gene amplification) described herein can be detected in a sample obtained from a mammal having a cancer, and can be used to determine whether or not the mammal will or is likely to respond to a particular cancer treatment. For example, amplification of any appropriate nucleic acid sequence (e.g., a MYC nucleic acid sequence, a ASXL1 nucleic acid sequence, a H3F3B nucleic acid sequence, a ERBB2 nucleic acid sequence, a CCND1 nucleic acid sequence, a CCNE1 nucleic acid sequence, a FGFR4 nucleic acid sequence, a KRAS nucleic acid sequence, a NOTCH4 nucleic acid sequence, a RAD51C nucleic acid sequence, and/or a RNF43 nucleic acid sequence) in a sample obtained from a mammal having a cancer, and can be used to determine whether or not the mammal will or is likely to respond to a particular cancer treatment. In some cases, the presence or absence of a gene amplification described herein in a cancer cell within a mammal having, or suspected of having, a cancer can be used to identify the mammal as being likely to respond to a particular cancer treatment (e.g., one or more PARP inhibitors). For example, a sample obtained from a mammal (e.g., a mammal having, or suspected of having, a cancer) can be assessed for the presence or absence of a MYC amplification. In some cases, the presence of a MYC amplification can be used to determine that the mammal will or is likely to respond to one or more PARP inhibitors to the mammal. In some cases, the absence of a MYC amplification can be used to determine that the mammal will not or is not likely to respond to one or more PARP inhibitors to the mammal.

In some cases, the presence of absence of one or more rearrangements (e.g., genome-wide rearrangements) described herein can be detected in a sample obtained from a mammal having a cancer, and can be used to determine whether or not the mammal will or is likely to respond to a particular cancer treatment. For example, rearrangement of any appropriate nucleic acid sequence (e.g., a MYC nucleic acid sequence, a YAP1 nucleic acid sequence, a MAML2 nucleic acid sequence, a IKZF2 nucleic acid sequence, a ERBB4 nucleic acid sequence, a CCND1 nucleic acid sequence, a SHANK2 nucleic acid sequence, a CCND1I nucleic acid sequence, a NF1 nucleic acid sequence, a TSC2 nucleic acid sequence, a FBXW7 nucleic acid sequence, a MLST8 nucleic acid sequence, and/or a FAM160A1 nucleic acid sequence) in a sample obtained from a mammal having a cancer, and can be used to determine whether or not the mammal will or is likely to respond to a particular cancer treatment. In some cases, the presence or absence of a fusion gene (e.g., YAP1-MAML2, IKZF2-ERBB4, SHANK2-CCND1, NF1-MYO1D, MLST8-TSC2, and/or FBXW7-FAM160A1) in a sample obtained from a mammal having a cancer can be assessed, and can be used to determine whether or not the mammal will or is likely to respond to a particular cancer treatment. In some cases, the presence or absence of a gene amplification described herein in a cancer cell within a mammal having, or suspected of having, a cancer can be used to identify the mammal as being likely to respond to a particular cancer treatment (e.g., one or more PARP inhibitors). For example, a sample obtained from a mammal (e.g., a mammal having, or suspected of having, a cancer) can be assessed for the presence or absence of one or more gene genome-wide rearrangements (e.g., rearrangements resulting a YAP1-MAML2 fusion gene and/or a IKZF2-ERBB4 fusion gene). In some cases, the presence of a YAP1-MAML2 fusion gene can be used to determine that the mammal will or is likely to respond to one or more PARP inhibitors to the mammal. In some cases, the absence of a YAP1-MAML2 fusion gene can be used to determine that the mammal will note or is not likely to respond to one or more PARP inhibitors to the mammal. In some cases, the presence of a IKZF2-ERBB4 fusion gene can be used to determine that the mammal will or is likely to respond to one or more PARP inhibitors to the mammal. In some cases, the absence of a IKZF2-ERBB4 fusion gene can be used to determine that the mammal will not or is not likely to respond to one or more PARP inhibitors to the mammal.

In some cases, the presence of absence of one or more modifications (e.g., activating modifications or inactivating modifications) described herein can be detected in a sample obtained from a mammal having a cancer, and can be used to determine whether or not the mammal will or is likely to respond to a particular cancer treatment. For example, a modification in any appropriate nucleic acid sequence (e.g., a PPP2R1A nucleic acid sequence, a PIK3CA nucleic acid sequence, a CDC73 nucleic acid sequence, a ERBB4 nucleic acid sequence, a EZH2 nucleic acid sequence, a MLH1 nucleic acid sequence, a TGFBR2 nucleic acid sequence, a SMAD3 nucleic acid sequence, a SMAD4 nucleic acid sequence, a ESR1 nucleic acid sequence, a CDK6 nucleic acid sequence, a NOTCH1 nucleic acid sequence, a STAG2 nucleic acid sequence, a ATRX nucleic acid sequence, a CDKN2A nucleic acid sequence, a CDKN2B nucleic acid sequence, a NF1 nucleic acid sequence, a NF2 nucleic acid sequence, a EZH2 nucleic acid sequence, a STK11 nucleic acid sequence, a TP53 nucleic acid sequence, a ARID1A nucleic acid sequence, a KRAS nucleic acid sequence, a APC nucleic acid sequence, and/or a CREBBP nucleic acid sequence) in a sample obtained from a mammal having a cancer, and can be used to determine whether or not the mammal will or is likely to respond to a particular cancer treatment. In some cases, the presence or absence of a gene amplification described herein in a cancer cell within a mammal having, or suspected of having, a cancer can be used to identify the mammal as being likely to respond to a particular cancer treatment (e.g., one or more MEK inhibitors and/or one or more PI3K inhibitors). For example, a sample obtained from a mammal (e.g., a mammal having, or suspected of having, a cancer) can be assessed for the presence or absence of one or more modifications in SMAD3 and/or SMAD4. In some cases, the presence of one or more inactivating modifications in SMAD3 and/or SMAD4 can be used to determine that the mammal will or is likely to respond to one or more MEK inhibitors to the mammal. In some cases, the absence of one or more inactivating modifications in SMAD3 and/or SMAD4 can be used to determine that the mammal will not or is not likely to respond to one or more MEK inhibitors to the mammal. For example, a sample obtained from a mammal (e.g., a mammal having, or suspected of having, a cancer) can be assessed for the presence or absence of one or more modifications in PPP2R1A. In some cases, the presence of one or more inactivating modifications in PPP2R1A can be used to determine that the mammal will or is likely to respond to one or more PI3K inhibitors to the mammal. In some cases, the absence of one or more inactivating modifications in PPP2R1A can be used to determine that the mammal will not or is not likely to respond to one or more PI3K inhibitors to the mammal. For example, a sample obtained from a mammal (e.g., a mammal having, or suspected of having, a cancer) can be assessed for the presence or absence of one or more modifications in PIK3CA. In some cases, the presence of one or more activating modifications in PIK3CA can be used to determine that the mammal will or is likely to respond to one or more PI3K inhibitors to the mammal. In some cases, the absence of one or more activating modifications in PIK3CA can be used to determine that the mammal will not or is not likely to respond to one or more PI3K inhibitors to the mammal.

A mammal having, or suspected of having, a cancer can be administered, or instructed to self-administer, one or more cancer treatments. For example, one or more cancer treatments can be administered to a mammal in need thereof. In some cases, a cancer treatment for a mammal having, or suspected of having, a cancer can be selected based, at least in part, on the presence or absence of one or more structural alterations described herein in one or more cancer cells within the mammal. For example, a sample obtained from a mammal having, or suspected of having, a cancer can be assessed for the presence or absence of one or more structural alterations described herein, and the presence or absence of one or more structural alterations described herein can be used to determine whether or not the mammal will or is likely to respond to a particular cancer treatment. For example, the presence or absence of one or more structural alterations described herein can be used to determine the responsiveness of a mammal having cancer to a particular cancer treatment, and a treatment option for the mammal (e.g., an individualized cancer treatment) can be selected, and, optionally, administered to the mammal. Individualized cancer treatments for the treatment of a mammal having a cancer (e.g., based, at least in part, on the presence or absence of one or more structural alterations described herein in one or more cancer cells within the cancer) can include any one or more (e.g., 1, 2, 3, 4, 5, 6, or more) cancer treatments. A cancer treatment can include any appropriate cancer treatment. In some cases, a cancer treatment can include administering one or more anti-cancer agents. An anti-cancer agent can be a chemotherapeutics such as an alkylating agent, a plant alkaloid, an antitumor antibiotic, an antimetabolite, a topoisomerase inhibitor, or an antineoplastic. An anti-cancer agent can be an immunotherapy such as a checkpoint inhibitor, an adoptive cell transfer, a monoclonal antibody, a treatment vaccine, or a cytokine. An anti-cancer agent can be a targeted therapy such as a small-molecule or a monoclonal antibody. An anti-cancer agent can be a hormone therapy such as an anti-antigen or an anti-estrogen. An anti-cancer agent can be a cellular therapy such as a stem cell transplant or an adoptive cell transfer. In some cases, a cancer treatment can include administering one or more PARP inhibitors to a mammal having cancer. For example, one or more PARP inhibitors can be administered to a mammal having cancer and identified as being likely to respond to one or more PARP inhibitors based, at least in part, on the presence or absence of one or more structural alterations described herein in one or more cancer cells within the cancer. Examples of PARP inhibitors include, without limitation, talazoparib (BMN-673), olaparib (AZD-2281), rucaparib (PF-01367338), niraparib (MK-4827), veliparib (ABT-888), CEP 9722, E7016, BGB-290, iniparib (BSI 201), and 3-aminobenzamide. Those of ordinary skill in the art will be aware of other suitable PARP inhibitors. In some cases, a cancer treatment can include administering one or more PI3K inhibitors to a mammal having cancer. For example, one or more PI3K inhibitors can be administered to a mammal having cancer and identified as being likely to respond to one or more PI3K inhibitors based, at least in part, on the presence or absence of one or more structural alterations described herein in one or more cancer cells within the cancer. Examples of PI3K inhibitors include, without limitation, GNE-493, wortmannin, demethoxyviridin, LY294002, hibiscone C, idelalisib, copanlisib, duvelisib, taselisib, perifosine, buparlisib, alpelisib (BYL719), umbralisib (TGR 1202), PX-866, dactolisib, CUDC-907, voxtalisib (SAR245409, XL765), ME-401, IPI-549, SF1126, RP6530, INK1117, pictilisib, XL147 (also known as SAR245408), palomid 529, GSK1059615, ZSTK474, PWT33597, IC87114, TG100-115, CAL263, RP6503, PI-103, GNE-477, and AEZS-136. Those of ordinary skill in the art will be aware of other suitable PI3K inhibitors. In some cases, a cancer treatment can include administering one or more MEK inhibitors to a mammal having cancer. For example, one or more MEK inhibitors can be administered to a mammal having cancer and identified as being likely to respond to one or more MEK inhibitors based, at least in part, on the presence or absence of one or more structural alterations described herein in one or more cancer cells within the cancer. Examples of MEK inhibitors include, without limitation, binimetinib (MEK162), trametinib (GSK1120212), cobimetinib (XL518), selumetinib, PD-325901, CI-1040, PD035901, and TAK-733. Those of ordinary skill in the art will be aware of other suitable MEK inhibitors. In some cases, a cancer treatment can include surgery. In some cases, a cancer treatment can include radiation treatment. In cases where two or more cancer treatments are administered, the two or more cancer treatments can be administered at the same time or independently.

As used herein, treating cancer includes reducing the number, frequency, or severity of one or more (e.g., two, three, four, or five) signs or symptoms of a cancer in a patient having a cancer. For example, treatment can reduce the severity of a cancer (e.g., can reduce the number of cancer cells or reduce the size of a tumor), reduce cancer progression (e.g., can reduce or prevent tumor growth and/or metastasis or can reduce the proliferative, migratory, and/or invasive potential of cancer cells), and/or reduce the risk of re-occurrence of a cancer in a subject having the cancer. In some cases, methods and materials provided herein can be used to reduce the number of cancer cells or reduce the size of a tumor in a mammal.

In some cases, when treating a mammal having a cancer as described herein, the treatment can increase survival of the mammal. For example, the treatment can increase progression-free survival of the mammal. For example, the treatment can increase overall survival of the mammal.

In some cases, when treating a mammal (e.g., human) having a cancer and identified as being likely to respond to one or more PARP inhibitors (e.g., based, at least in part, on the presence or absence of one or more structural alterations described herein) as described herein, the mammal can be administered, or instructed to self-administer, one or more PARP inhibitors to treat the mammal. For example, one or more one or more PARP inhibitors can be administered to a mammal in need thereof. For example, one or more PARP inhibitors (e.g., talazoparib (BMN-673), olaparib (AZD-2281), rucaparib (PF-01367338), niraparib (MK-4827), veliparib (ABT-888), CEP 9722, E7016, BGB-290, iniparib (BSI 201), and/or 3-aminobenzamide) can be administered to a mammal having cancer and identified as being likely to respond to one or more PARP inhibitors based, at least in part, on the presence of a MYC amplification in a cancer cell within the mammal. For example, BMN-673 can be administered to a mammal having an ovarian cancer including one or more cancer cells with the presence of a MYC amplification. In some cases, one or more PARP inhibitors (e.g., talazoparib (BMN-673), olaparib (AZD-2281), rucaparib (PF-01367338), niraparib (MK-4827), veliparib (ABT-888), CEP 9722, E7016, BGB-290, iniparib (BSI 201), and/or 3-aminobenzamide) can be administered as the sole active ingredient used to treat cancer. In some cases, one or more PARP inhibitors can be administered together with one or more additional agents/therapies other than PARP inhibitors used to treat cancer.

In some cases, when treating a mammal (e.g., human) having a cancer and identified as being likely to respond to one or more PI3K inhibitors (e.g., based, at least in part, on the presence or absence of one or more structural alterations described herein) as described herein, the mammal can be administered, or instructed to self-administer, one or more PI3K inhibitors to treat the mammal. For example, one or more one or more PI3K inhibitors (e.g., GNE-493, wortmannin, demethoxyviridin, LY294002, hibiscone C, idelalisib, copanlisib, duvelisib, taselisib, perifosine, buparlisib, alpelisib (BYL719), umbralisib (TGR 1202), PX-866, dactolisib, CUDC-907, voxtalisib (SAR245409, XL765), ME-401, IPI-549, SF1126, RP6530, INK1117, pictilisib, XL147 (also known as SAR245408), palomid 529, GSK1059615, ZSTK474, PWT33597, IC87114, TG100-115, CAL263, RP6503, PI-103, GNE-477, and/or AEZS-136) can be administered to a mammal in need thereof. For example, GNE-493 can be administered to a mammal having an ovarian cancer including one or more cancer cells with the presence of an inactivating modification in PPP2R1A in a cancer cell within the mammal. For example, GNE-493 can be administered to a mammal having an ovarian cancer including one or more cancer cells with the presence of an activating modification in PIK3CA in a cancer cell within the mammal. In some cases, one or more PI3K inhibitors (e.g., GNE-493, wortmannin, demethoxyviridin, LY294002, hibiscone C, idelalisib, copanlisib, duvelisib, taselisib, perifosine, buparlisib, alpelisib (BYL719), umbralisib (TGR 1202), PX-866, dactolisib, CUDC-907, voxtalisib (SAR245409, XL765), ME-401, IPI-549, SF1126, RP6530, INK1117, pictilisib, XL147 (also known as SAR245408), palomid 529, GSK1059615, ZSTK474, PWT33597, IC87114, TG100-115, CAL263, RP6503, PI-103, GNE-477, and/or AEZS-136) can be administered as the sole active ingredient used to treat cancer. In some cases, one or more PI3K inhibitors can be administered together with one or more additional agents/therapies other than PI3K inhibitors used to treat cancer.

In some cases, when treating a mammal (e.g., human) having a cancer and identified as being likely to respond to one or more MEK inhibitors (e.g., based, at least in part, on the presence or absence of one or more structural alterations described herein) as described herein, the mammal can be administered, or instructed to self-administer, one or more MEK inhibitors to treat the mammal. For example, one or more one or more MEK inhibitors (e.g., binimetinib (MEK162), trametinib (GSK1120212), cobimetinib (XL518), selumetinib, PD-325901, CI-1040, PD035901, and/or TAK-733) can be administered to a mammal in need thereof. For example, MEK162 can be administered to a mammal having an ovarian cancer including one or more cancer cells with the presence of an inactivating modification in SMAD3/4 in a cancer cell within the mammal. In some cases, one or more MEK inhibitors (e.g., binimetinib (MEK162), trametinib (GSK1120212), cobimetinib (XL518), selumetinib, PD-325901, CI-1040, PD035901, and/or TAK-733) can be administered as the sole active ingredient used to treat cancer. In some cases, one or more MEK inhibitors can be administered together with one or more additional agents/therapies other than MEK inhibitors used to treat cancer.

The invention will be further described in the following examples, which do not limit the scope of the invention described in the claims.

### EXAMPLES

### Example 1: Integrated Genomic, Epigenetic, and Expression Analyses of Ovarian Cancer Cell Lines

### Overall Approach

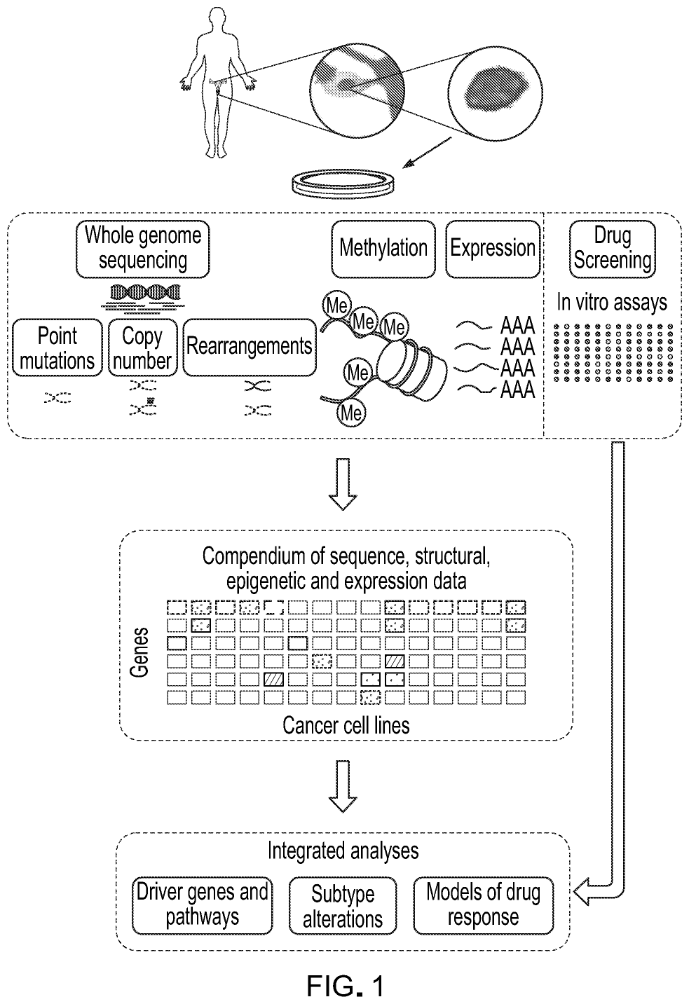

It was aimed to assemble a collection of ovarian cancer cell lines that would be representative of the different histological subtypes. These encompassed both publicly available as well as newly generated cell lines, ultimately comprising 19 serous, 9 clear cell, 3 mucinous, 2 undifferentiated, 2 endometrioid, 1 mixed, and 9 of unclassified subtypes (Table 1). The origin of the lines was confirmed using unique short tandem repeat (STR) analyses (Table 2). To identify sequence and structural changes in these ovarian cancer cell lines, next generation whole genome analyses were performed at an average coverage of 32× and 116.6 Gb per sample (Table 3). As matched normal DNA was not available for these samples, a set of 18 unmatched DNA samples from normal blood or lymphoblastoid cell lines from individuals of various ethnicities was also sequenced. Approaches were developed to focus on likely tumor-specific sequence and genome-wide structural changes, including amplifications, deletions and rearrangements. In parallel, genome-wide methylation analyses were performed and integrated with genomic and expression data in order to obtain a comprehensive molecular profile of these samples (FIG. 1).

### Sequence Analyses

A high sensitivity analysis of sequence alterations, including single base substitutions and small insertions and deletions, was performed for the exomes of these samples. Given the challenges of characterizing tumor-specific (somatic) changes in tumor samples without matched normal tissue, stringent bioinformatic approaches were developed to determine likely somatic mutations. Removal of common germline variants resulted in an average of 928 alterations per cell line exome, comprising 41,768 rare germline and somatic alterations. Six cell lines (two clear cell and one each of endometrioid, serous, unclassified, and mixed lineage) were hypermutated, having alterations in mismatch repair (MMR) genes MLH1, MSH2, MSH6, or PMS2 and six times as many sequence changes compared to those tumors that were MMR proficient (Table S4). To focus on likely somatic alterations involved in tumorigenesis, the sequence alterations in each cell line were analyzed and changes that have been previously detected in the coding genomes of other cancer patients were identified (see, e.g., Forbes et al., 2010 Nucleic Acids Research 38:D652-D657). Nonsense or frameshift inactivating mutations in a panel of tumor suppressor genes were also identified (Table 5). Through these analyses, 672 putative driver somatic mutations across 45 ovarian cell lines were discovered (Table 6).

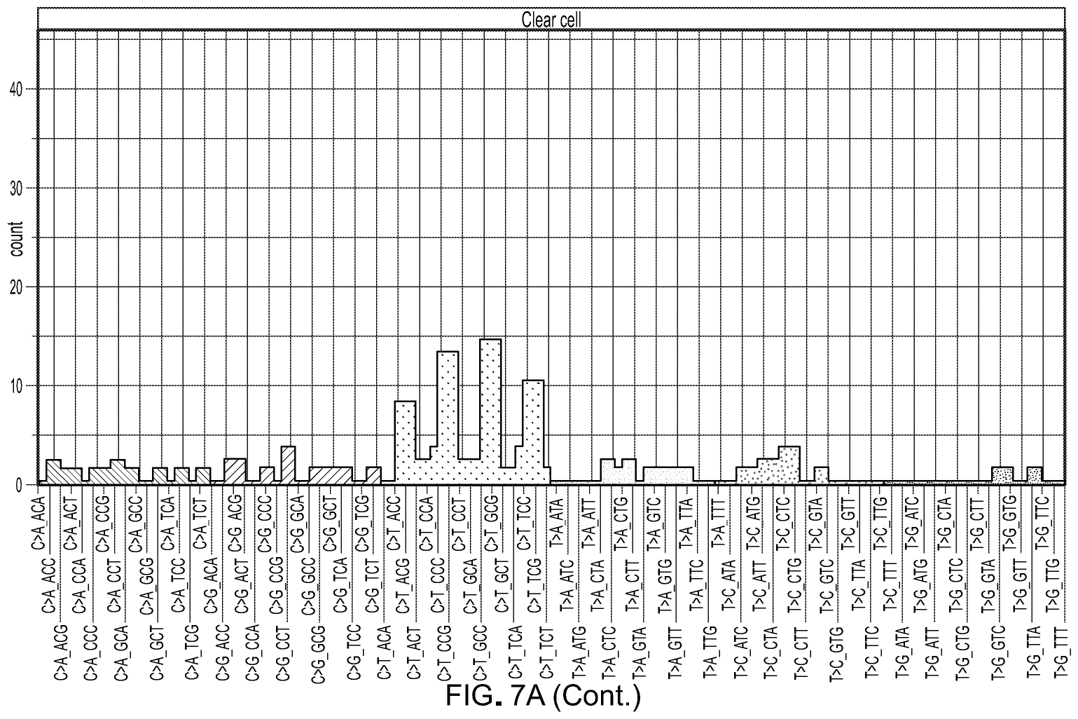

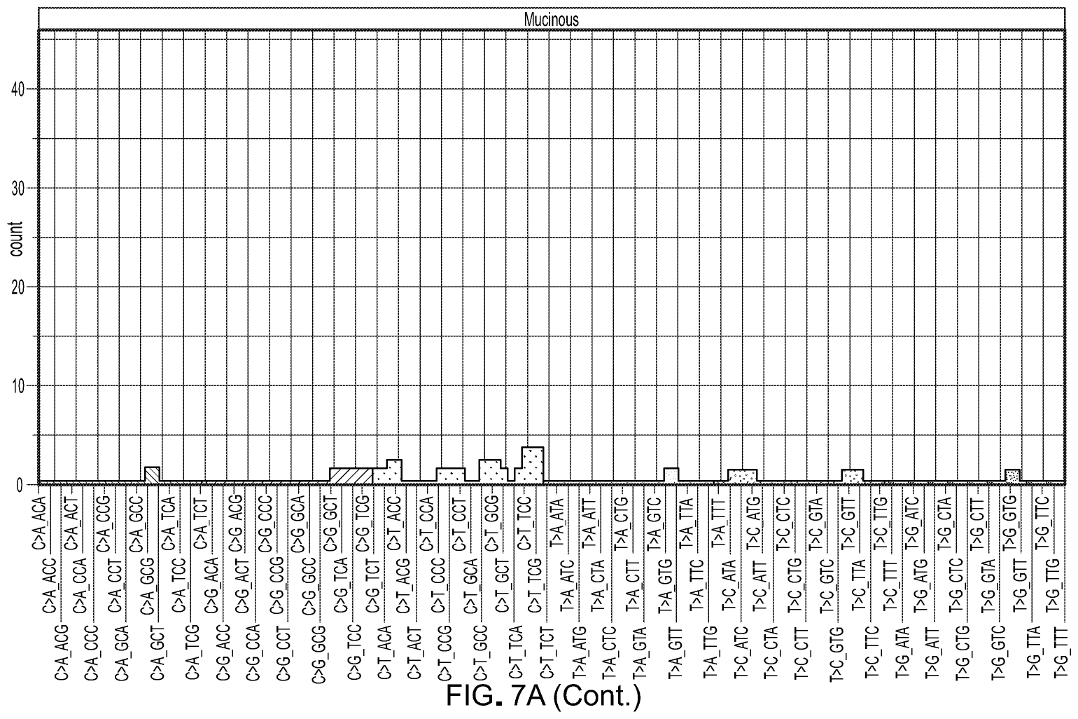

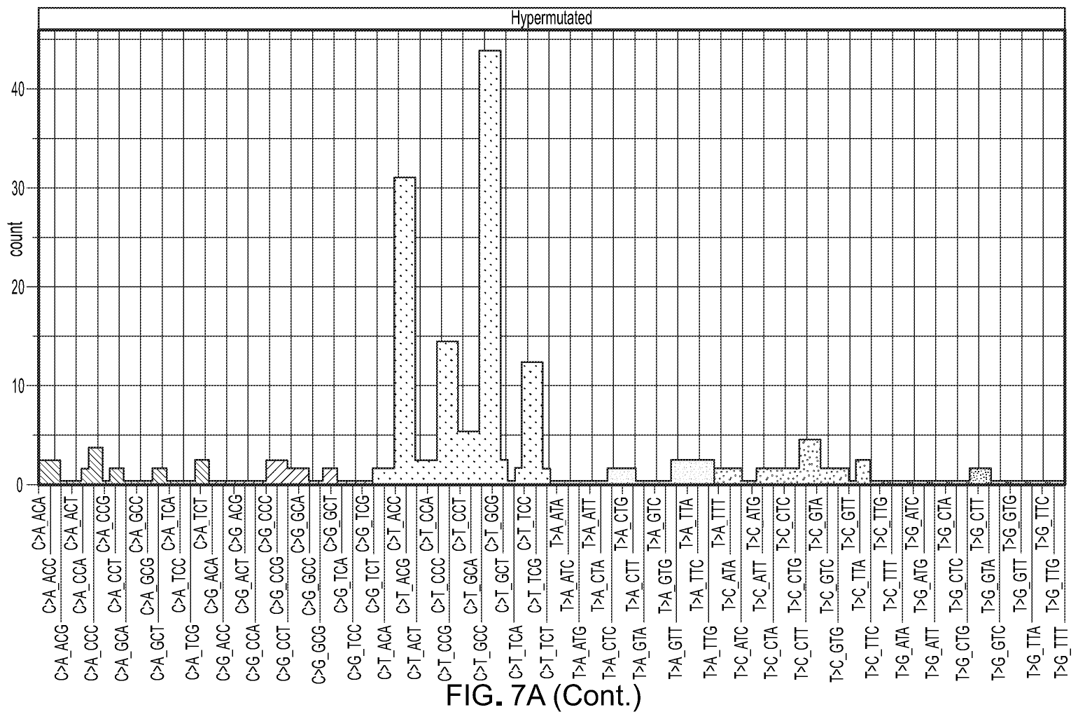

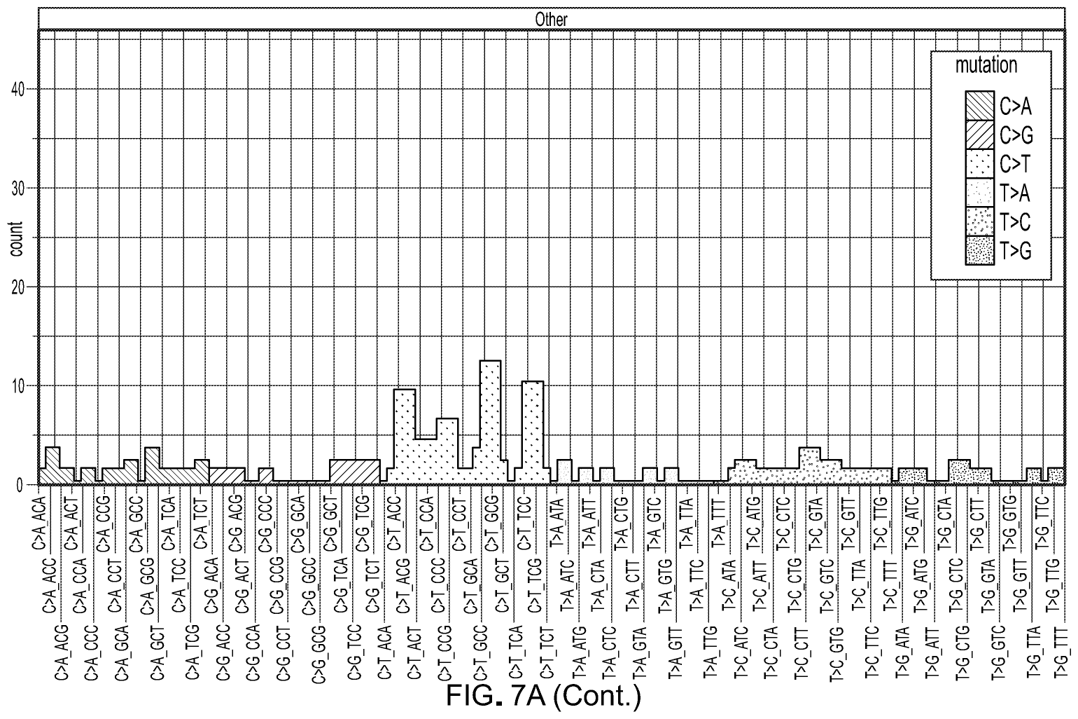

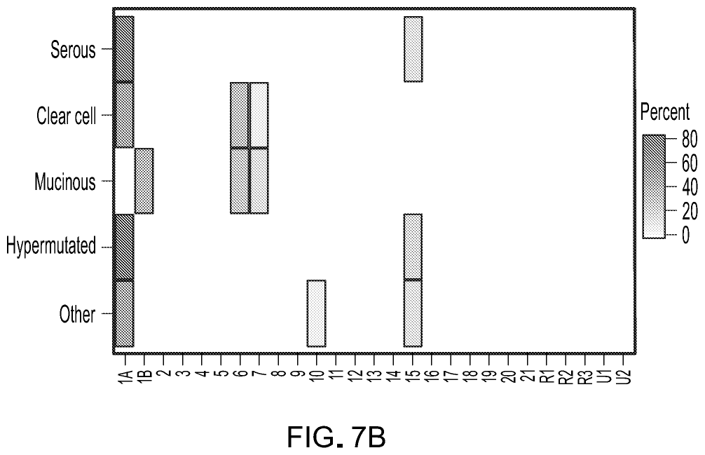

The most frequently mutated gene was the TP53 tumor suppressor gene (altered in 24 non-hypermutated and 3 hypermutated tumors). Excluding hypermutated samples, other genes frequently mutated included ARID1A (14 cancer cell lines), PIK3CA (6), SMAD4 (4), KRAS (3), APC (3), CREBBP (3), and PPP2R1A (3). Mutations were predominantly CpG transitions C→T or G→A (48%) followed by non-CpG transitions A↔G or C↔T (25%) (FIG. 7A). Analysis of mutation signatures aggregated by ovarian cancer subtypes revealed that serous, mucinous and undifferentiated tumor cell lines had an age-related signature. Clear cell and serous ovarian cancers also had a profile consistent with a mismatch repair associated mutation signature (FIG. 7B). Overall, both the compendium of mutated genes as well as mutation-associated signatures were representative of previous ovarian cancer genome analyses (Table 6).

### Structural Variant Analyses

Whole-genome sequence data were used to characterize copy number changes as well as rearrangements that may affect key driver genes. Existing approaches for whole genome analyses were first considered, including DELLY and LUMPY, but these typically use matched normal sequences to accurately identify tumor-specific rearrangements (see, e.g., Rausch et al., 2012 Bioinformatics 28:i333-i339; and Layer et al., 2014 Genome Biol. 15:R84). Given the multitude of tumor cell lines and other cancer specimens where matched normal DNA is not available, a framework was developed for structural variant detection called Trellis that could be used with tumor genome sequence data directly. Additionally, because many structural changes are linked genomically (i.e. an amplified gene has both copy number changes and rearrangements that can be located in multiple locations of the genome), it was aimed to connect the multiple changes that were related to individual genetic targets. The features of this approach include 1) detection of tumor-only structural changes through removal of germline and artifactual changes, 2) distinction of focal homozygous deletions and amplifications from larger structural changes, 3) connection of apparently disparate copy number regions using paired sequences in the same amplicons, 4) detection of homozygous and hemizygous deletions through copy number and rearrangement data, 5) confirmation of rearrangements using a stringent local re-alignment to detect and remove spurious paired read and split alignments, and 6) identification of in-frame rearrangements that would likely lead to gene fusions.

To implement the Trellis approach, low complexity sequences were excluded by mappability, as well as regions of germline copy number variants (CNVs) and rearrangements detected in the genomes of eighteen samples derived from normal blood cells. The remaining 2.7 Gb of the genome were divided into 1 kb bins and examined areas of increased read density (>2.75 fold) to identify copy number gains, and regions of decreased read density (<0.6 fold) to detect hemizygous or homozygous deletions greater than 2 kb using approaches similar to Digital Karyotyping (see, e.g., Wang et al., 2002 Proc Natl Acad Sci USA 99:16156-16161; and Leary et al., 2008 Proc Natl Acad Sci USA 105:16224-16229). Rearrangements were identified from atypical orientation or spacing of paired reads as well as split read alignments (see Methods).

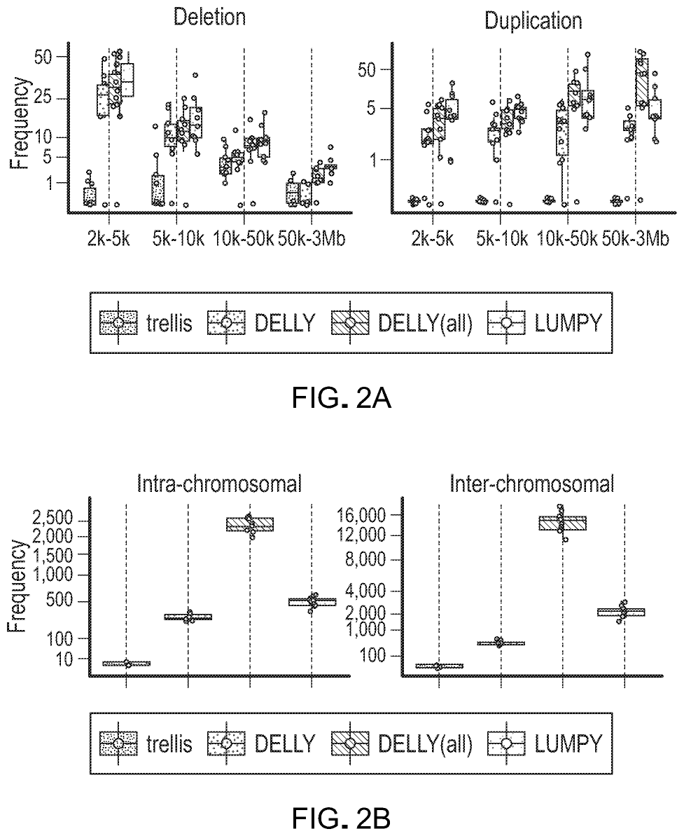

To evaluate the specificity of this approach in a set of non-tumor samples where very few somatic structural changes were expected, a leave-one-out cross-validation analysis among the 10 unmatched normal blood samples was used. Using Trellis, these analyses identified no focal high copy gains. On average, 5 hemizygous deletions (interquartile range 2-15) and one homozygous deletion (interquartile range 0-8) were identified in the normal samples (FIG. 2). Likewise, the average number of rearrangements observed per sample was three (interquartile range 0-6). These observations suggest a high specificity of our approach for detection of bona fide somatic alterations (mean specificity 0.97).

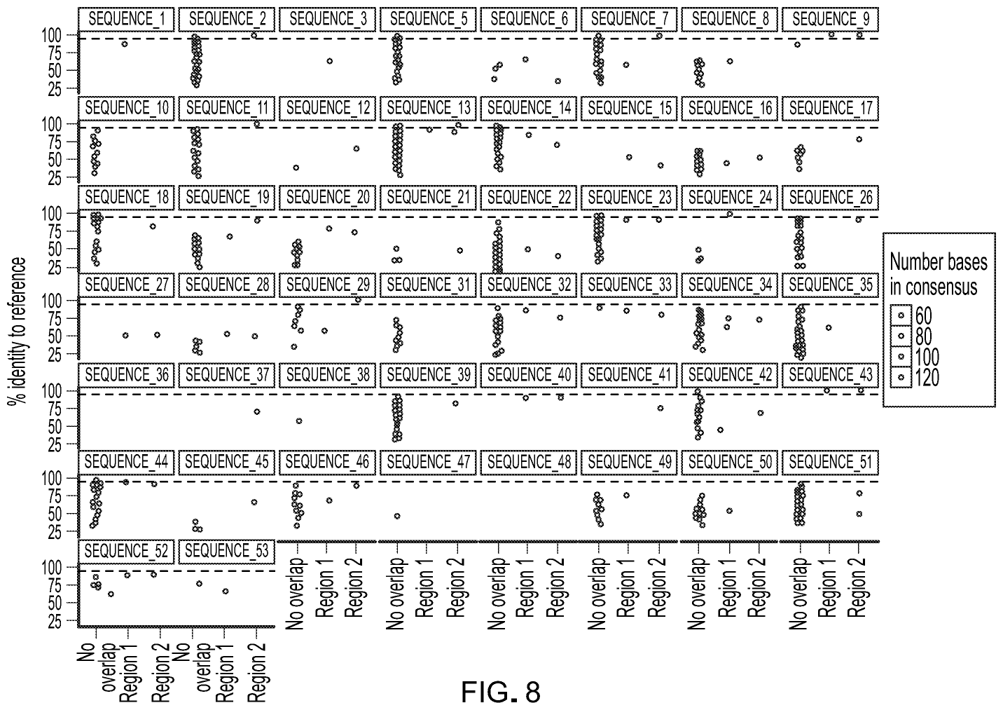

By contrast, analysis of normal samples with DELLY or LUMPY detected hundreds to thousands of structural changes in each normal DNA sample (FIG. 2). With DELLY and LUMPY, the average number of focal high quality copy number alterations was 13 and 21, respectively. The average number of intra- and inter-chromosomal rearrangements identified by DELLY was 297 and 433, respectively, and for LUMPY these were higher, at 511 and 2203, respectively. The number of alterations observed by DELLY using low-stringency settings was higher yet (FIG. 2). False positives for copy number changes appeared to largely be due to inclusion of single copy gains and losses, with neither DELLY nor LUMPY distinguishing hemizygous from homozygous losses or single copy gains from high copy amplifications. The source of the rearrangement false positives appeared to be largely the result of mapping artifacts due to low sequence complexity in putative rearrangements (FIG. 8).

To assess the sensitivity of this approach, 16 cell lines were sequenced using high coverage next generation sequencing of 111 genes comprising 585,216 bp. Computing the fold-change of read depth at these targeted regions, four high-copy amplifications with fold-change ≥6, nine low-copy amplifications with fold-change ≥3 and <6, and nine homozygous deletions were found. Trellis detected all four high-copy amplifications, including amplifications of AKT2, CCNE1, and KRAS. All nine regions identified as low copy amplifications by targeted sequencing were also determined to be low copy amplifications by Trellis, corroborating quantitative and qualitative characteristics of the amplifications. Similarly, all nine deletions discovered by targeted sequencing, comprising CDKN2A (8) and NF1 (1), were also characterized as homozygous deletions by Trellis. Overall, these analyses established that the Trellis approach had both high specificity and sensitivity for detection of structural alterations that are currently not possible with tumor-only samples using existing approaches.

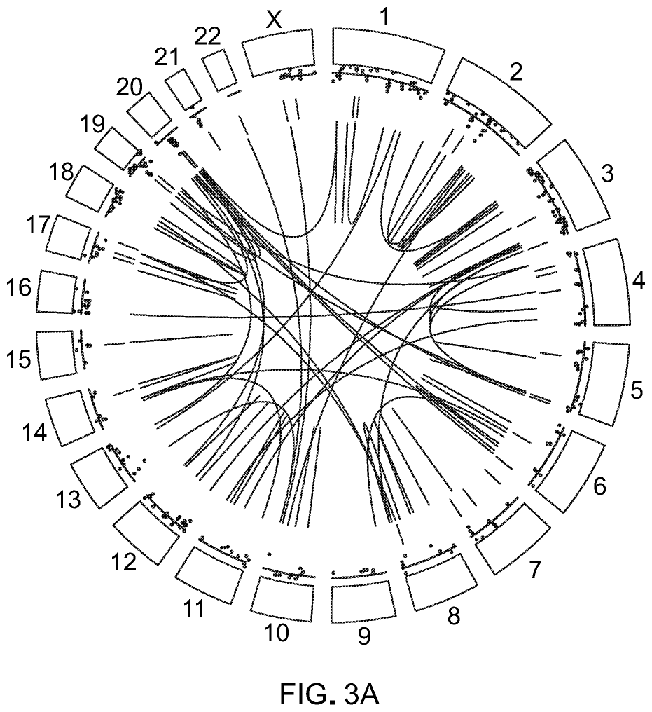

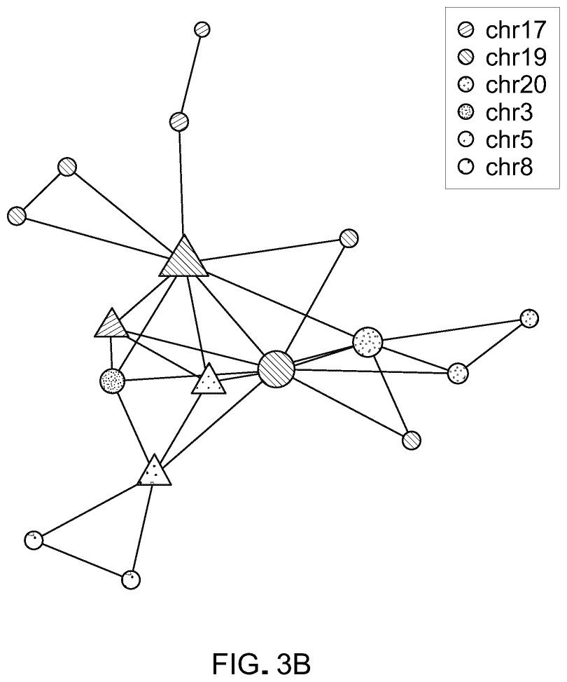

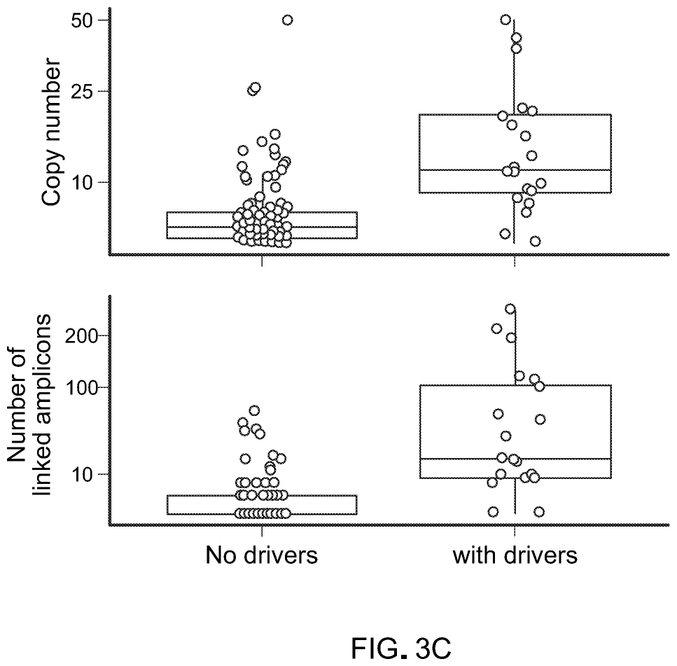

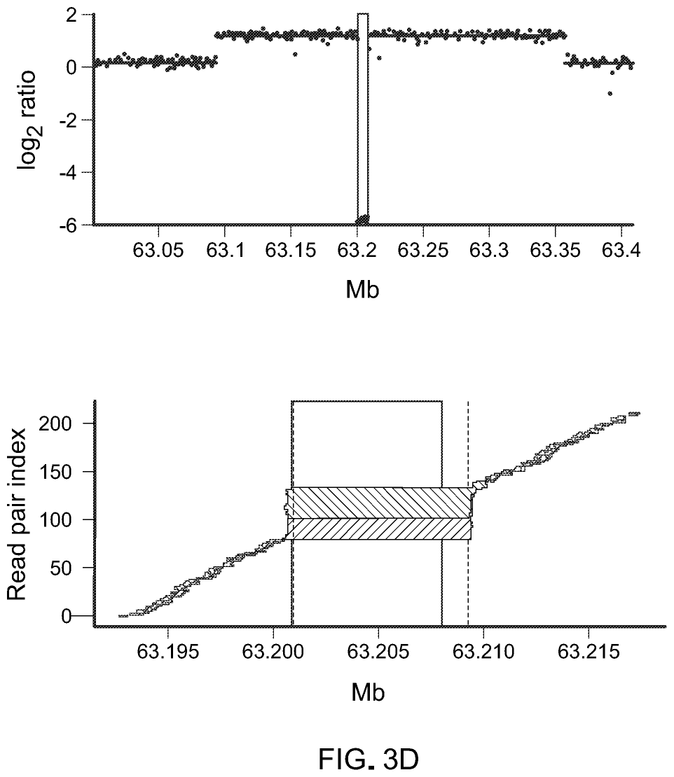

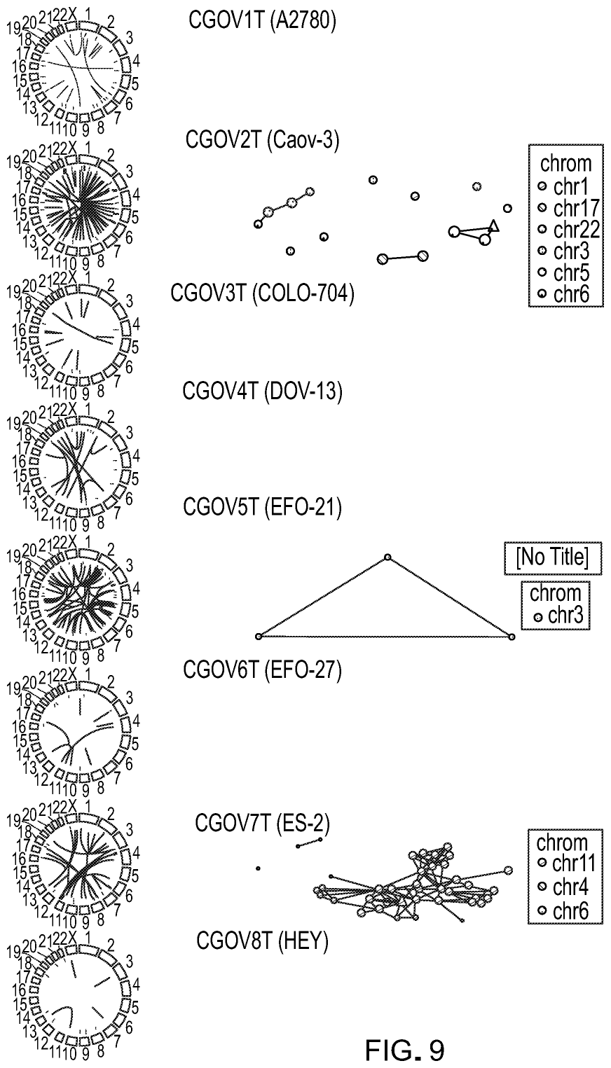

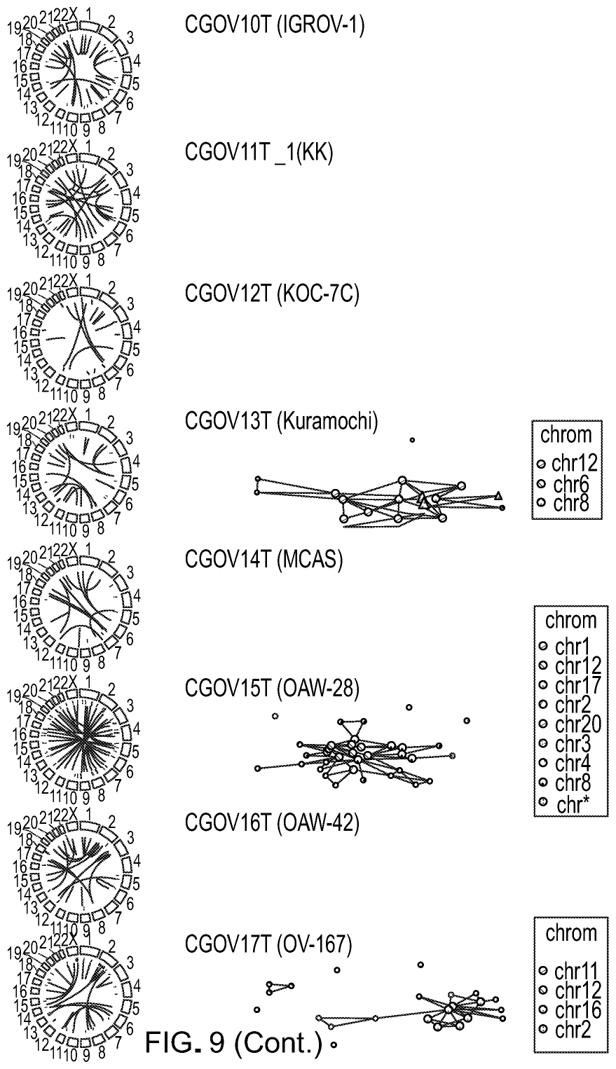

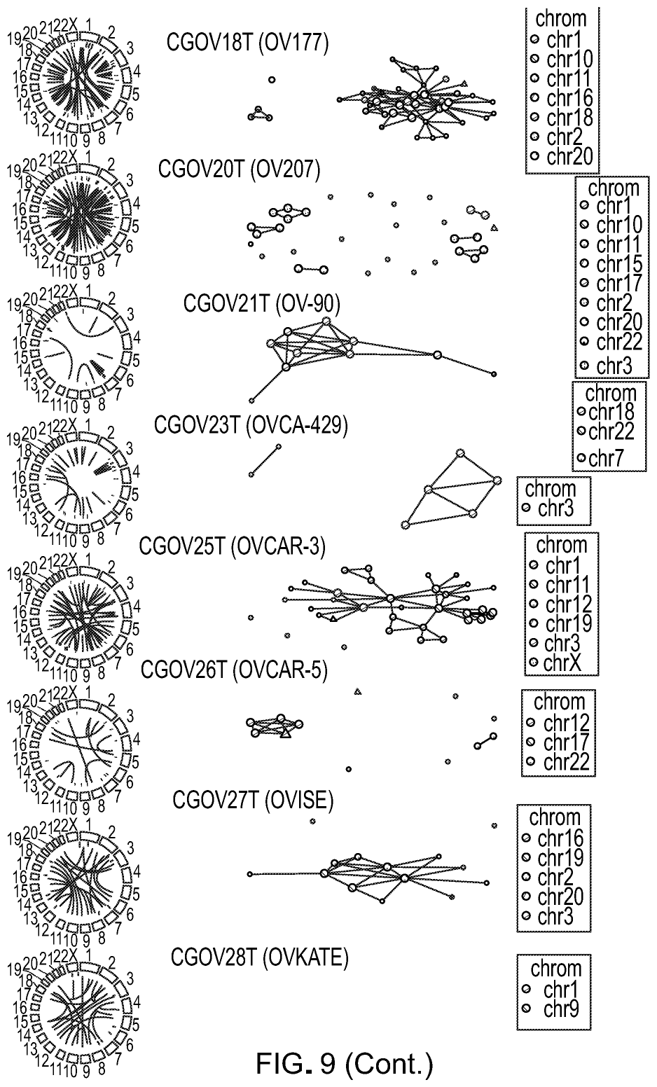

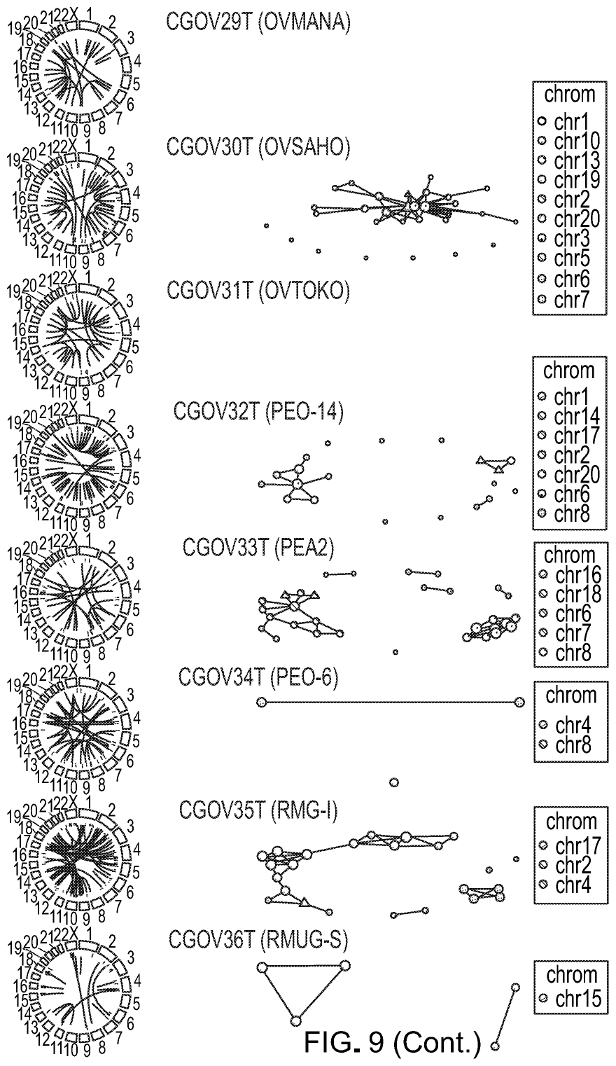

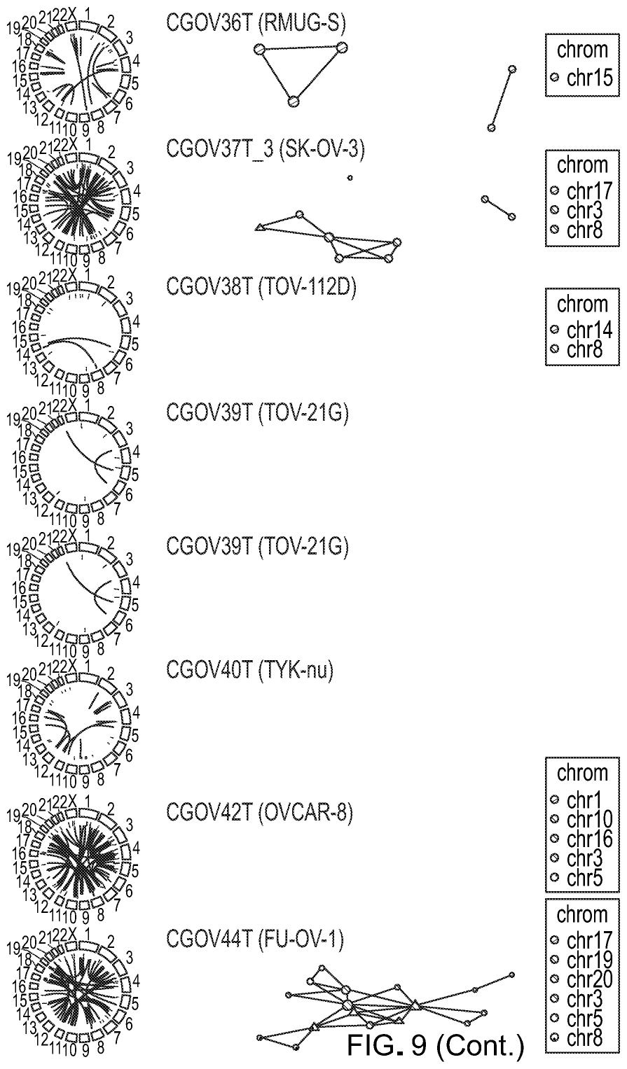

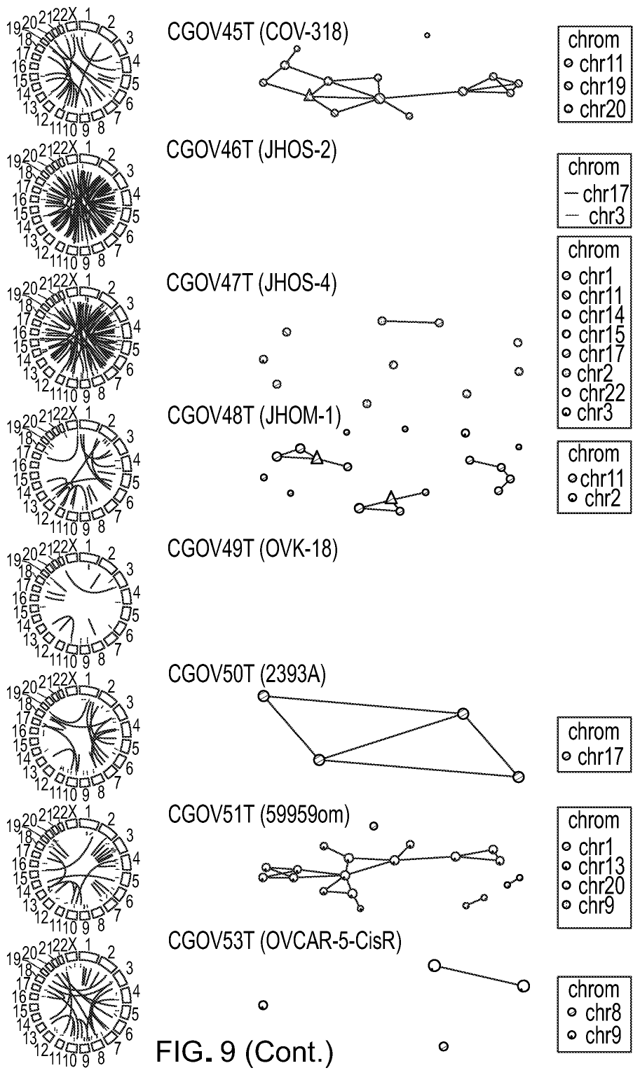

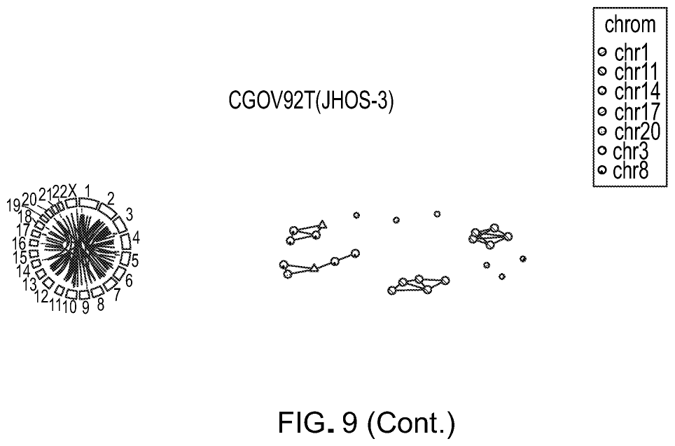

Linked amplicons: The analysis of amplifications was focused to regions smaller than 3 Mb that were present at >2.75 fold compared to the modal genome copy number. An analysis of the 45 ovarian cancer samples identified 538 focal amplicons, or an average of 12 amplicons per tumor (Table 7). As multiple amplicons within the same tumor may be derived from an amplification of a single target gene localized to different chromosomal regions, the possibility that amplicons may be linked was examined. Using our paired read whole genome analyses, it was found that reads at the edges of many amplicons were linked with aberrant spacing and/or orientation with respect to the reference genome. In order to identify links between apparently distant amplicons, these were visualized as undirected graphs where the nodes were amplicons and edges between amplicons were defined by multiple paired reads aligned to both genomic locations (e.g., FIGS. 3A and 3B). The analyses discovered 57 amplicon groups from the 538 amplicons across the ovarian tumor cell lines. Among tumors with at least one amplicon, the median number of amplicon groups was two and the median number of amplicons within an amplicon group was four (interquartile range 2-9). The majority of cell lines (15/28) with an amplicon group contained known driver genes. As an example, cell line ES-2 had 41 apparent amplicons, but through this approach it was determined that 38 of the amplicons were linked to a single group that contained the CCND1 driver gene (FIG. 9). Both the copy number and number of connections between amplicons was significantly higher for amplicon groups containing known drivers compared to amplicon groups without known drivers (FIG. 3C).

Driver genes that were amplified in two or more cell lines as part of amplicon groups that have previously been observed in ovarian cancer included well known oncogenes such as MYC (4), ERBB2 (2), CCND1 (2), CCNE1 (2), FGFR4 (2), and KRAS (2). Interestingly, amplifications of cancer driver genes were identified that have not been previously appreciated in ovarian cancer, including epigenetic regulator ASXL1 (2), H3 histone family member H3F3B (2), NOTCH family receptor NOTCH4 (1), repair and recombination paralog RAD51C (1), and ubiquitin ligase RNF43 (1). Several of these genes have been observed as being part of larger structural alterations in recent TCGA high grade serous ovarian carcinoma analyses (see, e.g., Network, 2011 Nature 474:609-615) but have not been identified as target genes in those cases of these alterations.

Overall, these analyses greatly simplified the observed amplification events and revealed that many focal amplicons would not have been associated with driver genes had they not been linked in specific amplicon groups. The observed amplicons were consistent with previously detected genes in ovarian cancer, but genes not previously implicated in this disease were also detected.

Deletions: A combination of stringent analyses of segmented read depth and aberrant read pair spacing to was used identify homozygous and hemizygous deletions. As deletions may occur in the germline, we removed deletions that were in or near structural alterations observed in the normal lymphoblastoid controls in order to identify those deletions that were most likely to be somatic. These analyses revealed 674 hemizygous+, 41 overlapping hemizygous+, 286 homozygous, and 263 homozygous+deletions, where ‘+’ denotes evidence for deletion supported by rearranged read pairs in addition to read depth (FIG. 3D and Table S8). Deletion breakpoints with rearranged read pairs were more precise (typically within 100 bp), while deletions without rearranged read pairs had a resolution of 1-5 kb. Homozygous deletions from segmentation analyses were included even if these were without rearranged read pairs as these could have been missed in read pair analyses due to the limited mappability at one or both deletion breakpoints. The median number of homozygous and hemizygous deletions per tumor was 10.5 (interquartile range 8-16) and 11.0 (interquartile range 6-18), respectively. Genes that were recurrently deleted included cell cycle regulators CDKN2A (9) and CDKN2B (8), tyrosine kinase receptor ERBB4 (5), neurofibromin genes NF1 (3) and NF2 (3), transcriptional regulator CDC73 (2), polycomb-group repressor EZH2 (2), and serine/threonine kinase STK11 (2) (Table S8), of which CDKN2A, NF1, NF2, and STK11 have been previously reported to be altered in high grade serous ovarian carcinomas (see, e.g., Network, 2011 Nature 474:609-615; and Huang et al., 2012 BMC Medical Genomics 5:47). Genes that have been implicated through somatic deletion in other tumors but that had not been previously implicated in ovarian cancer include CDC73, ERBB4, EZH2, MLH1 as well as TGF beta pathway members TGFBR2, SMAD3, and SMAD4, estrogen receptor ESR1, cell cycle kinase CDK6, notch receptor NOTCH1, cohesin member STAG2, and epigenetic regulator ATRX (Table S8). In a fashion similar to amplifications, several of these genes have been observed as being part of larger structural alterations in recent TCGA high grade serous ovarian carcinoma analyses (Network, 2011 Nature 474:609-615) but have not been identified as target genes in those cases or other histologic subtypes. The absence or low frequency of such alterations in previous studies may in part reflect the challenges of identifying bona fide deletions through existing approaches in primary tumors.

Other recurrent deletions included genes encompassing large genomic regions (>1 Mb) that were more likely to be affected by structural alterations, including a member of the low density lipoprotein receptor family LRP1B (7), fragile histidine triad involved in purine metabolism FHIT (11), a member of the short-chain dehydrogenases/reductases protein family WWOX(15), and the deacetylase MACROD2 (7). FHIT and WWOX occur in fragile sites, are often deleted in cancers, and some evidence suggests they encode putative tumor suppressors (Ohta et al., 1996; Zöchbauer-Müller et al., 2000; Roy et al., 2011; Aldaz et al., 2014). LRP1B deletion has been associated with chemotherapy resistance in high grade serous ovarian cancers and is a putative tumor suppressor (Cowin et al., 2012). Because of their proximity to CDKN2A, the methylthioadenosine phosphorylase MTAP and the transcription factor DMRT1 are commonly co-deleted with CDKN2A (Zhang et al., 1996), and use of compounds exploiting the loss of MTAP has been proposed as a potential therapeutic avenue (Marjon et al., 2016) for tumors with CDKN2A deletions.

Rearrangements and fusions: We next examined structural rearrangements that were not associated with segmental copy number changes. 850 inter-chromosomal and 2339 intra-chromosomal rearrangements were detected (Table S9). The median per sample of inter- and intra-chromosomal rearrangements was 16 (interquartile range 5-31) and 39 (interquartile range 17-63), respectively, with many of these rearrangements involving inversions (median of 8 and 7, respectively).

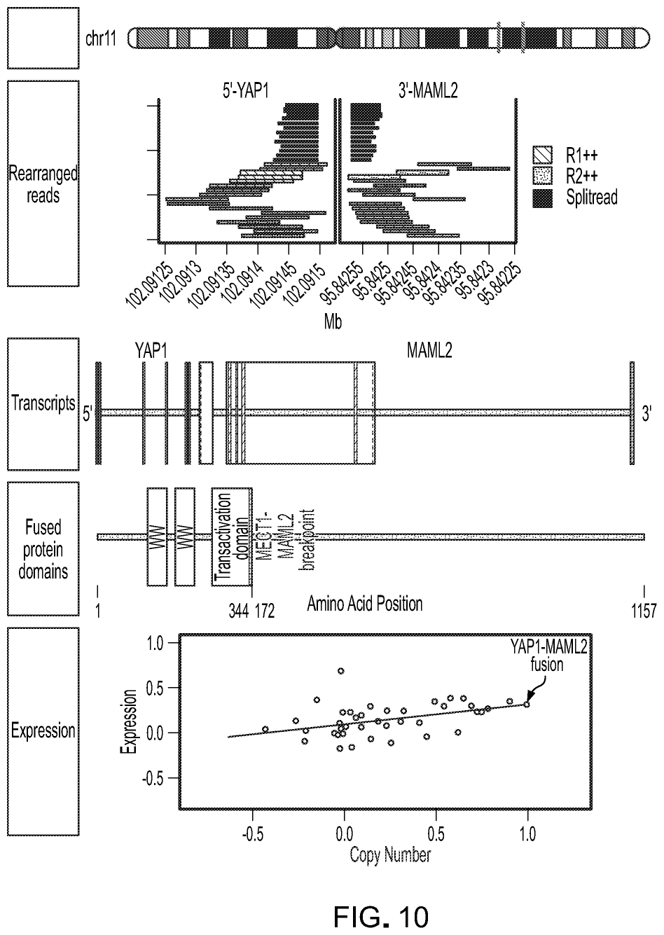

Among rearrangements for which the sequence junction was within the intron or exon of a gene, 290 in-frame fusions of two genes were detected (Table 10). Several of these in-frame fusions have not been observed in ovarian cancer but have been previously reported in other cancers. For example, YAP1-MAML2 has been reported in nasopharyngeal carcinoma and salivary cancers (Tonon et al., 2003; Coxon et al., 2005; Valouev et al., 2014), IKZF2-ERBB4 has been reported in T cell lymphomas (Boddicker et al., 2016), and fusions involving CCND1 were identified in a patient with leukemic mantle cell lymphoma (Gruszka-Westwood et al., 2002). This study discovered the YAP1-MAML2 fusion in cell line ES-2 after exon 6 of YAP1 and before exon 2 of MAML2, preserving the transactivation domain of MAML2 and its likely role in Notch signaling (FIG. 10). The breakpoint in the amino acid sequence of MAML2 is the same as reported in nasopharyngeal carcinoma and salivary gland cancers (amino acid 172) (Tonon et al., 2003; Coxon et al., 2005; Valouev et al., 2014).

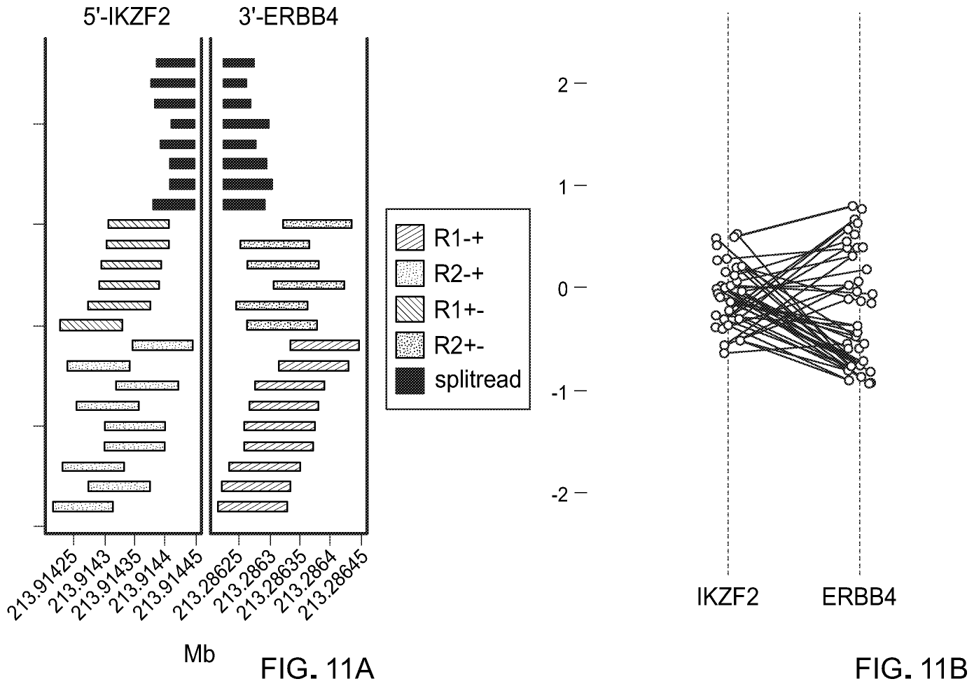

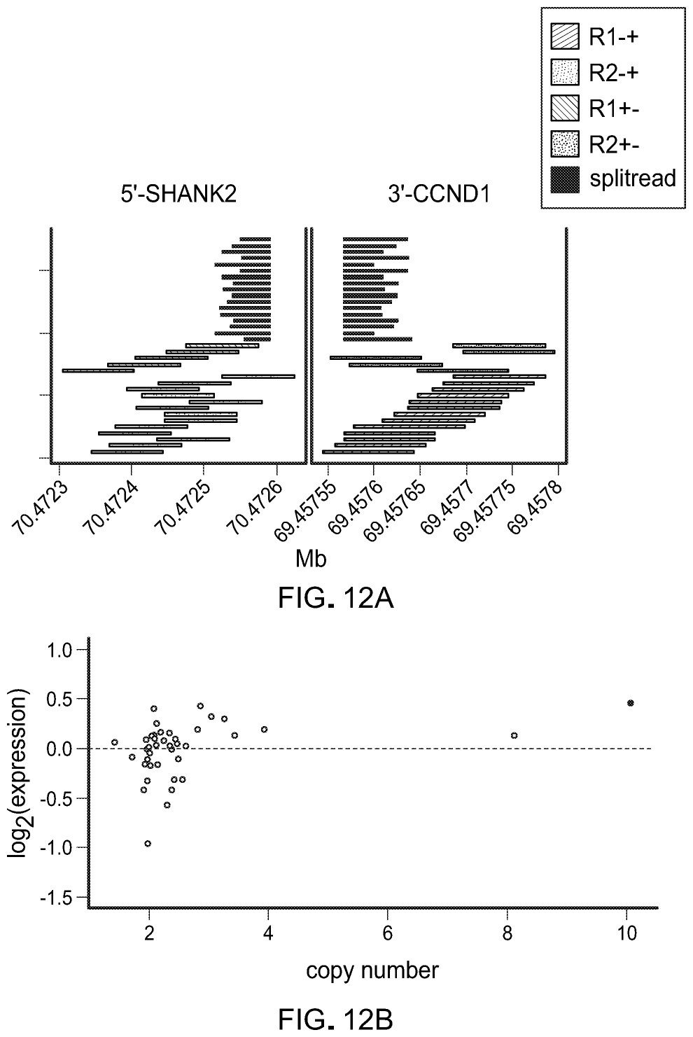

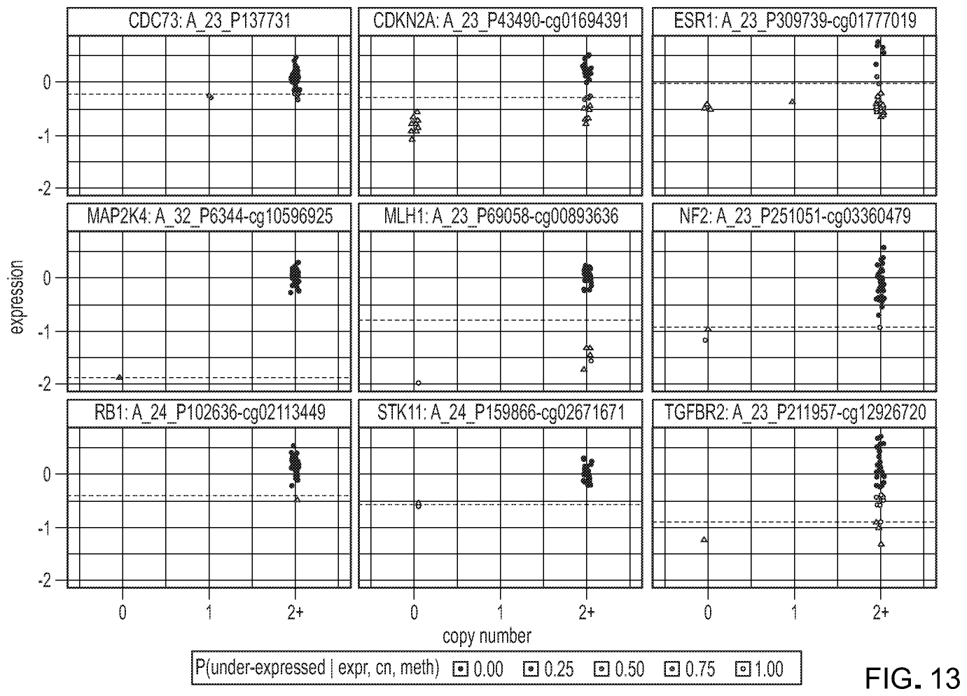

The IKZF2-ERBB4 fusion identified in ovarian tumor KK involves the first 3 exons of IKZF2 and exons 2-27 of ERBB4, a member of the epidermal growth factor receptor (EGFR) family. This IKZF2-ERBB4 junction is nearly identical to that reported by Boddicker et al. in T-cell lymphoma and mucinous lung adenocarcinoma, involving the same exons of ERBB4 and leaving the ERBB4 kinase domain intact (Boddicker et al., 2016). Gene expression analyses indicated that the ERBB4 transcript, including the fusion transcript, was over-expressed (FIG. 11). ERBB4 over-expression has been associated with resistance to platinum-based therapy in ovarian serous carcinomas (Saglam et al., 2017), suggesting a potentially important role for this translocation event for therapeutic selection. In ovarian tumor ES-2, CCND1 was amplified and also participated in a fusion where the promoter of SHANK2 was linked to the coding region of CCND1 (FIG. 12). An amplification and fusion involving CCND1 has been previously identified in a patient with leukemic mantle cell lymphoma (Gruszka-Westwood et al., 2002). Additional gene fusions not previously observed in ovarian cancer involved the negative regulator of the RAS pathway NF1, the tumor suppressor regulating mTORC1 signaling TSC2, and the member of the F-box protein family FBXW7. The fusion of NF1 (NF1-MYO1D) occurred after the first exon of this gene and would be expected to disrupt its function, consistent with its tumor suppressive role Network (2011). Similarly, the fusion of MLST8-TSC2 would be expect to result in a TSC2 protein lacking the first 373 amino acids, disrupting the key region of interaction with TSC1 (Guertin and Sabatini, 2005). As detailed below, the fusion of full-length FBXW7 to the promoter of FAM160A1 was also likely deleterious, due to decreased expression under the new promoter. For all of the predicted nine fusions involving at least one gene previously identified in other cancer fusions, all novel sequence junctions were independently validated using PCR and Sanger sequencing and a recently developed droplet digital PCR approach (Cumbo et al., 2018) (FIG. 13).

### Epigenetic and Expression Analyses

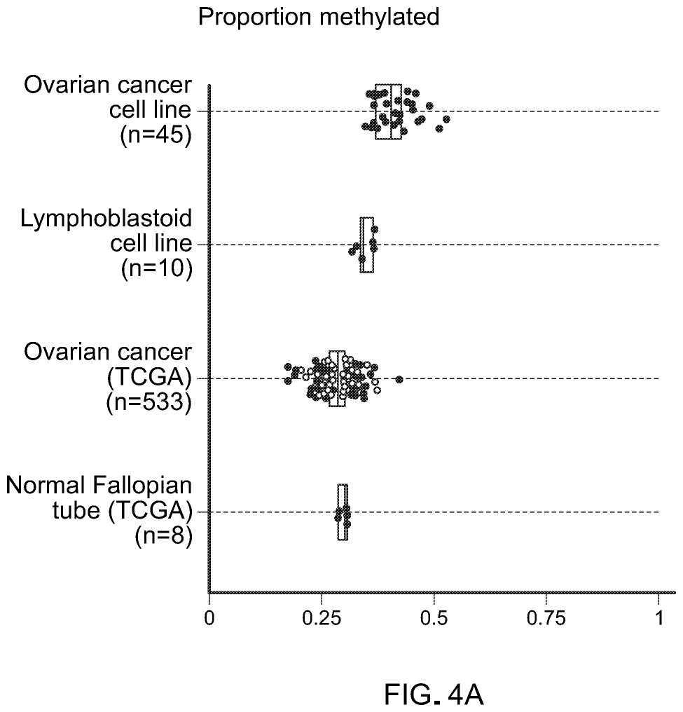

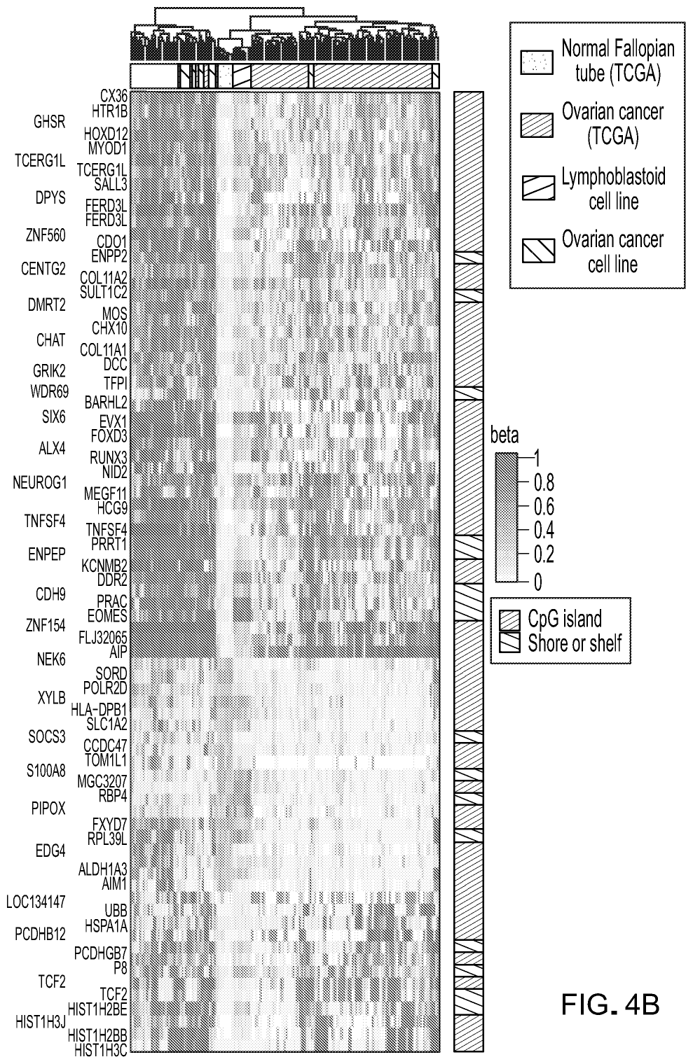

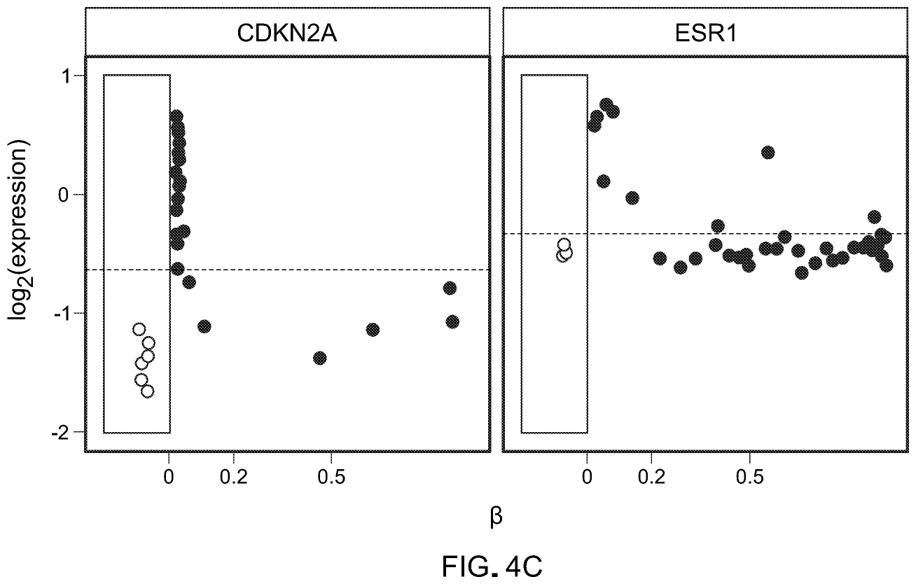

Genome-wide methylation profiles were examined in order to evaluate the role of epigenetic alterations in these ovarian cancer cell lines. Analyses of over 850,000 methylation sites were performed using Infinium MethylationEPIC arrays. Methylation levels were evaluated at individual CpG sites within gene promoter regions (±1500 bp upstream of the transcription start site) or within individual genes. Methylation levels in the ovarian cell lines were compared to methylation levels in the normal lymphoblastoid cells, as well as to 8 TCGA normal fallopian tissue and 533 TCGA ovarian cancers. Among the 18,619 CpG probes shared by the Infinium HumanMethylation27 BeadChip array (27,578 probes) and the MethylationEPIC array, we estimated the proportion of methylated CpG sites as the fraction of CpG probes with β>0:3. It was found that the overall proportion of methylated CpG sites in the lymphoblastoid (median 0.35) and ovarian cell lines (median 0.41) was higher than the proportion in fallopian tissues (median 0.30) and ovarian cancers (median 0.29) (FIG. 4A). To examine methylation profiles of the cell lines at individual CpG sites in the broader context of ovarian cancer methylation profiles, 96 genes were identified that were differentially methylated between normal fallopian tissue and 100 randomly sampled TCGA ovarian tumors (FIG. 4B). While both the lymphoblastoid cell lines and the ovarian cancer cell lines were excluded from the probe selection procedure, the normal lymphoblastoid cell lines were more highly correlated to the normal fallopian tissues while the ovarian cancer cell lines were more correlated to the TCGA ovarian cancers. Taken together, these analyses indicate that the ovarian cell lines retain epigenetic profiles of genes commonly methylated in ovarian cancer and that the methylation of these genes is unlikely to be related to growth in culture.

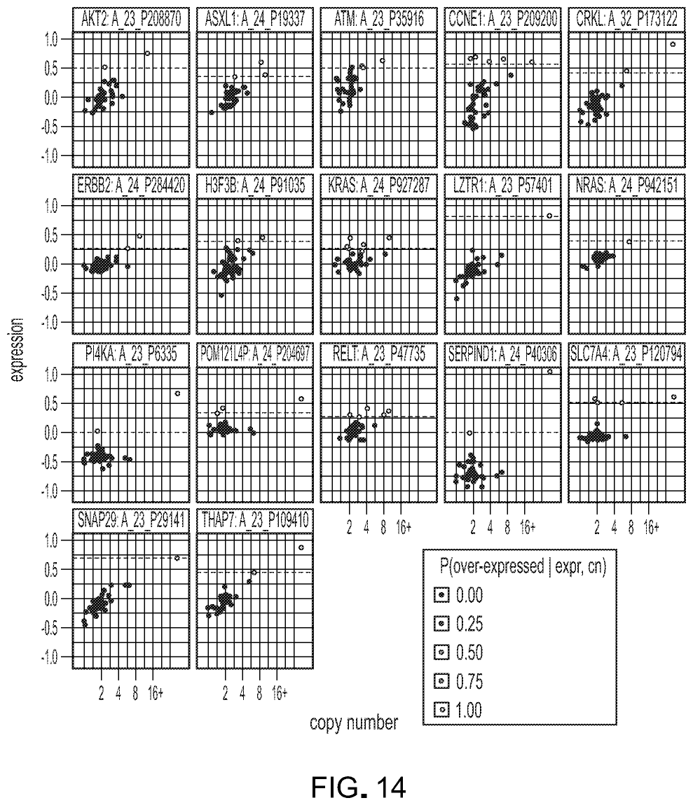

The genomic and epigenetic analyses were integrated with expression data previously obtained for these cell lines through the Agilent 44K array (see, e.g., Konecny et al., 2011 Clinical Cancer Research 17:1591-1602). It was assessed whether specific genes affected by deletions or other structural changes in some tumors may be silenced through methylation and low expression in others. Among genes that were methylated or deleted, expression analyses revealed lower expression for many of these genes. Cell lines RMG-I and IGROV-1 both had hemizygous deletion and loss of expression of CDC73. Of the 13 drivers homozygously deleted in at least one tumor, five genes, including CDKN2A and ESR1, displayed loss of expression and concomitant promoter methylation in additional ovarian cancers (FIGS. 4C and 14). When promoter methylation and underexpression were considered, the fraction of tumors with alterations in CDKN2A more than doubled from 23% to 55%, highlighting the multiple mechanisms by which CDKN2A function can be compromised. Similarly, MLH1 was mutated in a single case, but was mutated and/or underexpressed in an additional seven cancers. For ESR1, the inactivating methylation is thought to be associated with age and has been previously observed in both ovarian cancers and ovarian cancer cell lines (see, e.g., Imura et al., 2006 Cancer Letters 241:213-220; and Wiley et al., 2006 Cancer 107:299-308). Lower expression also resulted from abnormal fusion of non-adjacent promoters to the full coding sequence of target genes. In OVCAR-8, the fusion of the promoter of FAM160A1 with the full length FBXW7 gene resulted in dramatically decreased expression of FBXW7 (FIG. 11).

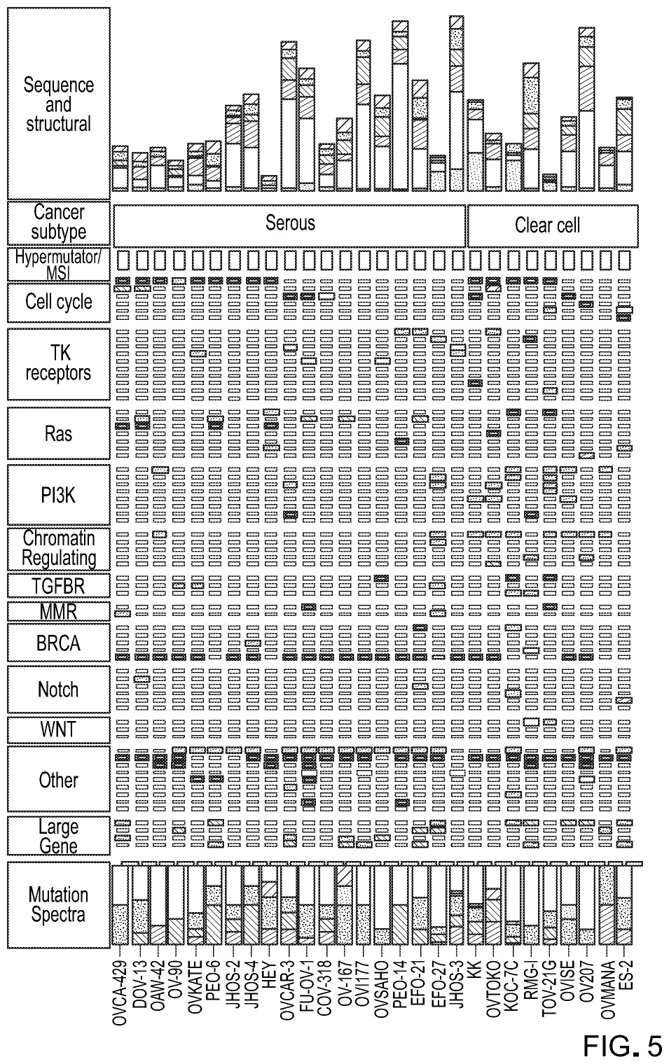

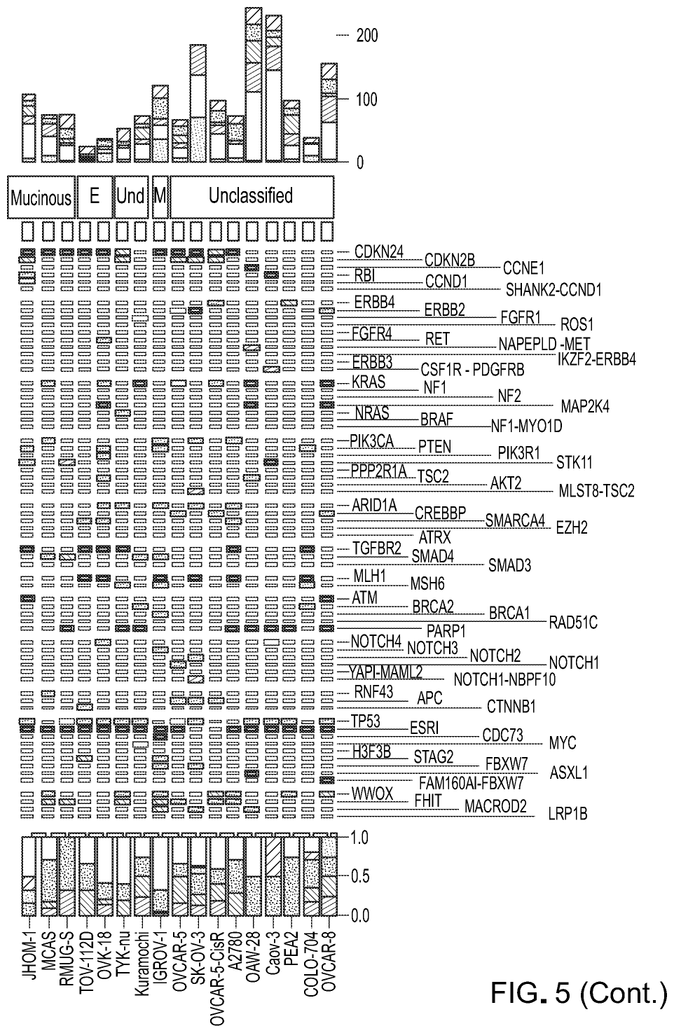

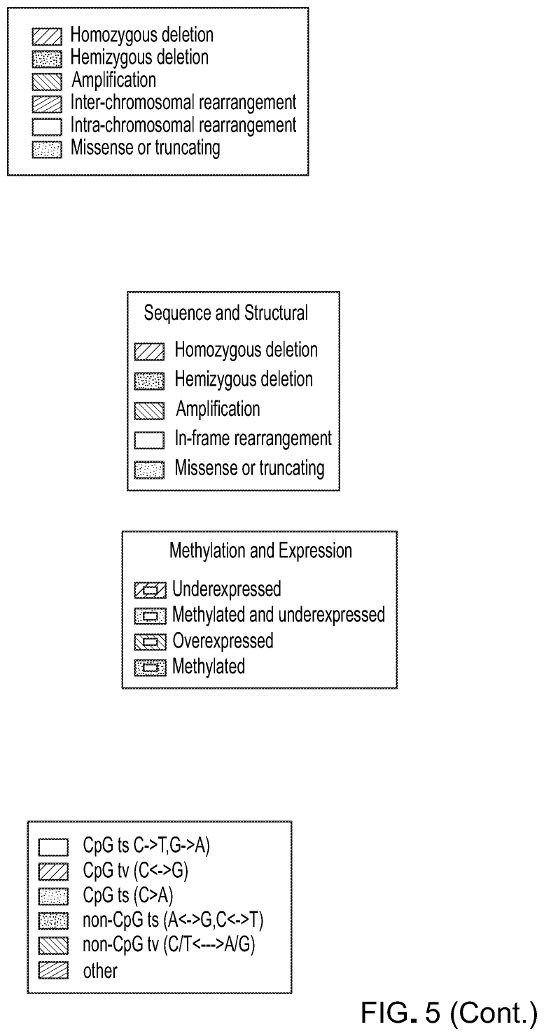

It was also examined the possibility of increased expression for genes with structural changes. 17 genes with focal amplification were identified in one or more cancer cell lines and evidence of bimodal expression across the samples analyzed. For these genes, 20 of the 22 tumors (91%) with focal amplification also had increased expression (Figure S9). Genes associated with amplification and fusion had particularly high expression, suggesting that the combination of genetic alterations led to increased overall transcription of these genes. The amplification of CCND1 and fusion in SHANK2-CCND1 fusion in sample ES-2 increased the expression of CCND1 relative to other ovarian cell lines without the amplification and fusion (FIG. 12). The YAP1-MAML2 fusion which was also duplicated in the same sample resulted in expression of MAML2 that was higher than 85% of the other ovarian cancer cell lines (FIG. 10). For driver genes that were amplified, it was examined whether additional tumors may be identified with increased expression of these genes. It was found increased expression of CCNE1, ERBB2, KRAS and AKT2 in eight additional cases without genomic alterations in these genes (FIGS. 5 and S9). These analyses indicate the importance of integrated genomic, epigenetic, and expression analyses and have resulted in an expansion of the number of tumors with alterations in key driver genes. These observations also highlight the functional consequences of genomic and epigenomic alterations in human cancer at the RNA level.

Combining sequence and structural variants with methylation and differential gene expression, it was found that nearly all ovarian cancer subtypes had alterations in cell cycle, chromatin remodeling, DNA repair, RAS, Notch, PI3K, or TGFB signaling pathways (FIG. 5). Alterations in the cell cycle pathway genes, including CDKN2A, were the most common with one or more alterations in 60-70% of the three most represented subtypes (serous, adenocarcinoma, and clear cell). Chromatin modifications occur in (5/7) (71%) of the clear cell subtypes but in only 2/21 (11%) of the serous samples. Evidence of mutual exclusivity was see between CDKN2A, CCNE1, and RB1 within the cell cycle pathway, but not mutual exclusivity between cell cycle and KRAS pathways, underscoring that clonal selection often involves multiple drivers regulating distinct molecular processes.

### Sensitivity and Resistance to Pathway Inhibitors

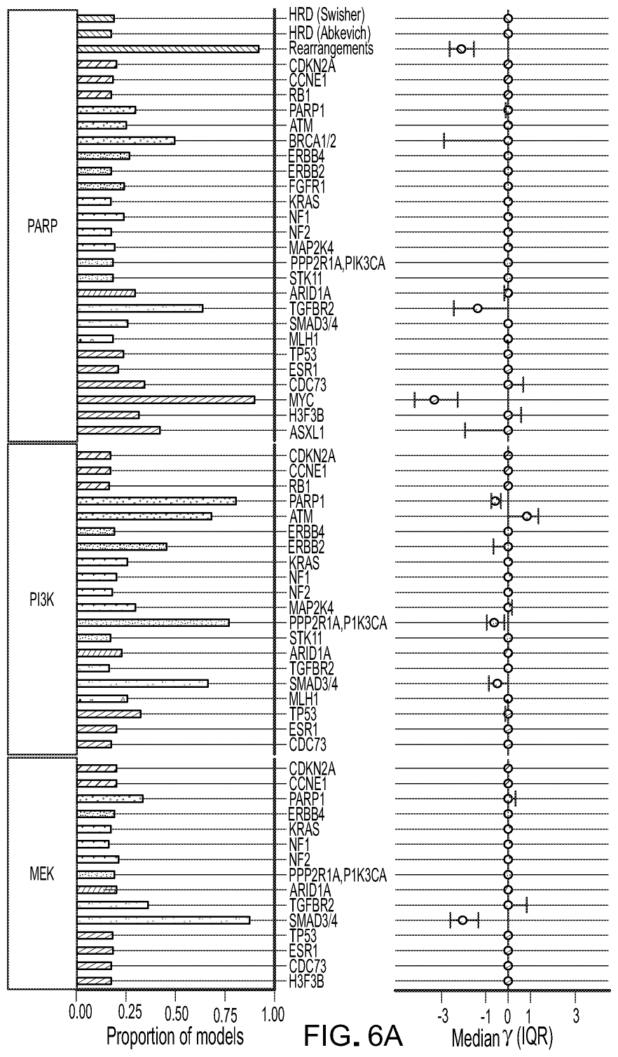

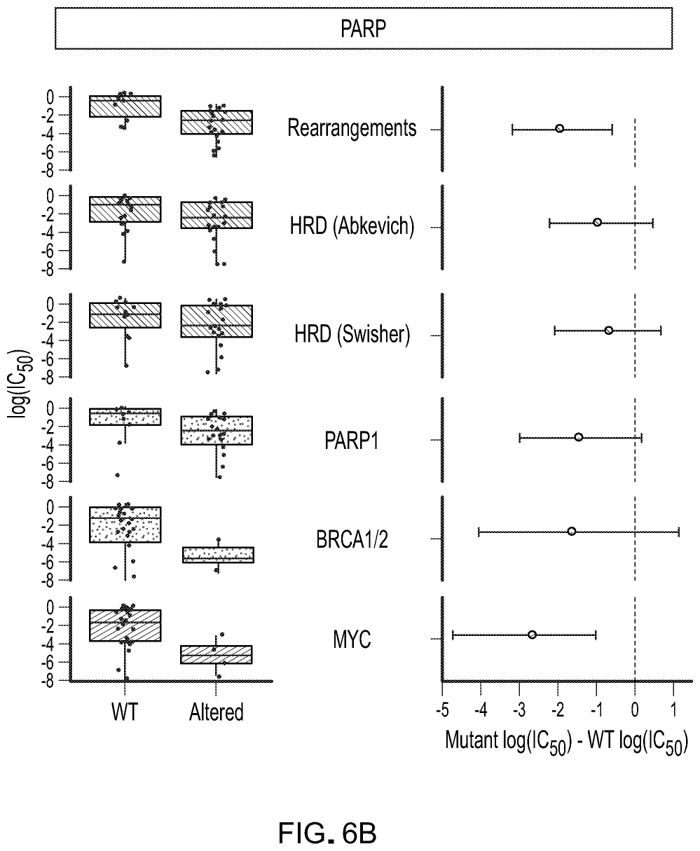

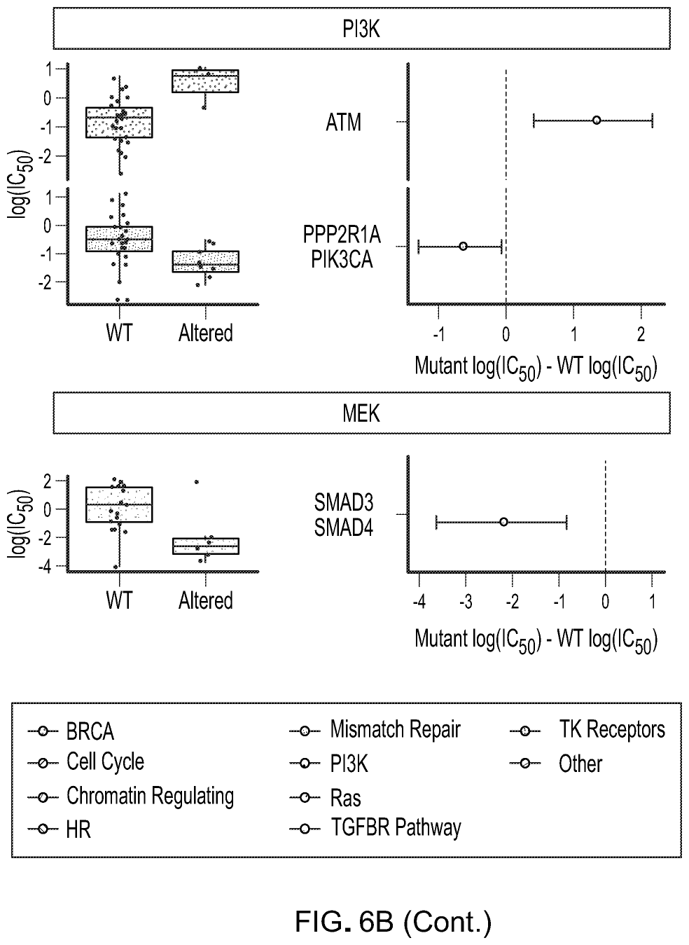

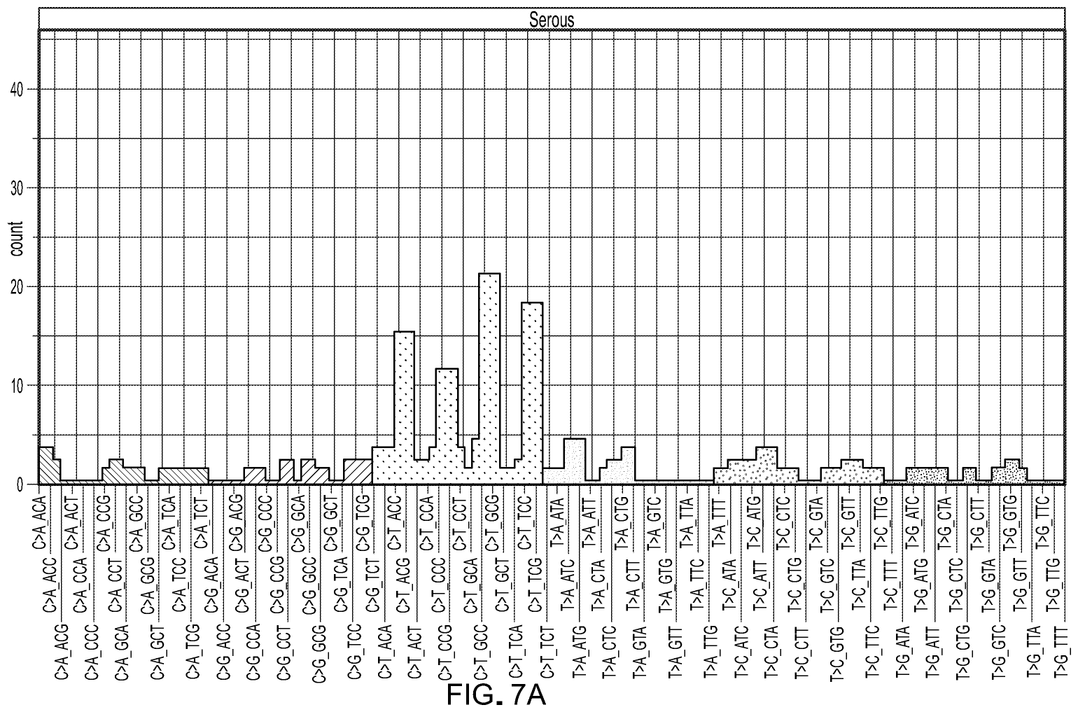

To begin to understand the relationship between genomic, epigenetic and expression alterations and response to pathway inhibitors, a screening platform was developed for evaluating cellular proliferation in the presence of candidate therapeutic agents. As an example of the analyses that can be performed and the genotype-phenotype connections that can be obtained, IC₂₀, IC₅₀, and IC₈₀ were measured after seven days of incubation for three inhibitors, GNE-493, BMN673, and MEK162, targeting PI3K, PARP, and MEK proteins, respectively (Table 11). Aggregating the molecular information from multiple platforms to the gene level, analyses was limited to genes that were altered in three or more of the 45 cell lines. Alterations that tend to be mutually exclusive between cell lines were combined, including genes in the PI3K pathway (PIK3CA and PPP2R1A) and the genes in the TGFBR pathway (SMAD3 and SMAD4). As tumors with homologous recombination deficiencies (HRD) have been known to be sensitive to PARP inhibitors, covariates summarizing the extent of genome-wide structural alterations for the PARP inhibitor BMN673 were additionally added. A priori it was hypothesized that most alterations would not modulate response to the targeted inhibitors. Implementing a Bayesian model averaging approach to variable selection as has been considered for other biomarkers (see, e.g., Viallefont et al., 2001 Statistics in Medicine 20:3215-3230; Neto et al., 2014 Pacific Symposium on Biocomputing. Pacific Symposium on Biocomputing: 27-38; and Meisner et al., 2018 Biomarker research 6:3), a positive prior probability that the coefficient for each gene is exactly zero was specified. Given the genes or combination of genes and structural variant summaries, the space of possible single- and multi-variate models for logIC₅₀ was explored by Markov Chain Monte Carlo. Relevant posterior summaries available for each inhibitor include the probability that the regression coefficient is non-zero and the posterior distribution of the regression coefficients. This approach was used to focus on those features that were present in at least half of the models as these had a higher probability of being predictive for drug response (FIG. 6A).

For PARP inhibition by BMN673, analyses revealed that the number of genome-wide rearrangements and amplification of MYC were important predictors of drug sensitivity (FIG. 6). Importantly, the two cell lines with inactivating BRCA1/2 mutations as well as the HRD score were applied through our whole genome analyses, and PARP1 expression showed a trend towards increased sensitivity to PARP inhibition but were not statistically significant (FIG. 6). It was found that amplification of MYC or an increase in the number of genome-wide rearrangements, including inversions and intra-chromosomal rearrangements, were significantly associated with sensitivity to this therapy, appearing in 94% of the single and multi-variate models. It was estimated the difference of the mean log IC₅₀ between the group of tumors with alterations in these features and the group of tumors without such changes, revealing a 93% (90% CI: 99%-64%) and 86% (90% CI, 96%-43%) increased sensitivity to PARP inhibition for cell lines with MYC amplification and increased rearrangements, respectively (FIG. 6). Although other genomic signatures and PARP1 expression have been suggested as biomarkers for PARP sensitivity (Nik-Zainal et al., 2016 Nature 534:47-54), MYC amplification and rearrangements have not been previously identified as markers of PARP sensitivity in serous and endometrioid ovarian cancers. Taken together, these results suggest that alterations of common drivers along with large-scale structural alterations in ovarian cancer may identify tumors with high sensitivity to this therapy.

For inhibition of the PI3K pathway by GNE-493, mutations of PPP2R1A or PIK3CA appeared in more than 75% of the models evaluated. Cancer cell lines with mutations in PARP1 or PPP2R1A had a 66% increased sensitivity to GNE-493 (FIG. 6). The results suggest that PI3K inhibitors counter the loss of PI3K pathway regulation from inactivating mutations of PPP2R1A and activating mutations of PIK3CA.

For the MEK pathway, mutations or deletions in SMAD3 or SMAD4 were predictive of IC₅₀ levels in response to the inhibitor MEK-162. These were selected in more than 85% of the models and resulted in an increased sensitivity of 89% to this therapy. The results are show that loss of SMAD4 can lead to activation of Smad-independent MEK/ERK pathway signaling and that inhibition of this pathway with MEK inhibitors can reverse tumorigenic effects.

### Experimental Procedures

### Cell Lines and Growth Analyses

Cell lines were obtained from multiple sources (Table 1). Cells were plated into 24-well tissue culture plates at a density of 2×10⁵ to 5×10⁵ cells per well and grown in cell-line-specific medium without or with increasing concentrations of their respective drugs (ranging between 0.001 and 10 μm/L).

Cells were counted on day 7 using an automated cell viability assay (Vi-CELL XR Cell Viability Analyzer, Beckman Coulter, Fullerton, Calif., USA), a video imaging system that uses an automated trypan blue exclusion protocol. Both adherent and floating viable cells were counted for treatment and control wells. Growth inhibition (GI) was calculated as a percentage of untreated controls. The log of the fractional GI was then plotted against the log of the drug concentration and the IC₅₀ values were interpolated from the resulting linear regression curve fit (CalcuSyn; Biosoft, Ferguson, Mo., USA). Experiments were performed thrice in duplicate for each cell line.

### STR Analyses

Genomic DNA from all cell lines was PCR amplified using a Geneprint 10 System (Promega, Madison, Wis.) that contains eight short tandem repeat loci plus Amelogenin, a gender determining marker. The PCR amplification was carried out in a GeneAmp PCR System 9700 following the manufacturer's protocol. The PCR products were electrophoresed on a ABI Prism 3730x1 Genetic Analyzer using Internal Lane Standard 600 (Promega) for sizing. Data was analyzed using GeneMapper v. 4.0 software (Applied Biosystems, Foster City, Calif.). STR profiles (JHU) for these cell lines were compared to external STR profiles, including those described elsewhere (see, e.g., Korch et al., 2012 Gynecol. Oncol. 127:241-248; COSMIC (v83, cancer.sanger.ac.uk/cosmic); the RIKEN BioResource Center (jove.com/institutions/AS-asia/JP-japan/20278-riken-bioresource-center); and Yu et al. 2015 Nature 520:307-311) (Table 2). The average percent similarity between JHU STRs and external STRs was 98%. An external STR was not available for 5 cell lines.

### Whole Genome Next Generation Sequencing

DNA was extracted from cell lines using a QIAamp DNA Blood Mini QIAcube Kit (Qiagen Valencia, Calif.). In brief, the samples were incubated in proteinase K for 16 hours before DNA extraction. DNA purification was performed using the QIAamp DNA Blood Mini QIAcube kit following the manufacturer's instructions (Qiagen, Valencia, Calif.). Genomic DNA from tumor samples were used for Illumina TruSeq library construction (Illumina, San Diego, Calif.) according to the manufacturer's instructions. Paired-end sequencing resulting in 100 bases from each end of the fragments was performed using Illumina HiSeq2000 instrumentation.

### PCR and Sanger Sequencing

PCR and Sanger sequencing confirmed the presence of fusion candidates generated by Trellis. Primers were designed 200 bp on either side of the junction and are shown in Table 13. Primers were purchased from IDT (Coralville, Iowa, USA). Primers were purified by desalting and upon arrival, primers and probes were resuspended to 100 μM in IDTE (10 mM Tris, pH 8.0; 0.1 mM EDTA) buffer and stored at −20° C. Using the primers specific for each fusion, PCR amplification was performed in a 50 μL reaction volume in quadruplicate, consisting of 10 μL of 5× Phusion buffer, 1 μL of 10 mM dNTP, 2.5 μL of each primer at 10 μM, 0.5 μL of HotStart Phusion and 10 ng of cell line DNA. PCR was performed using a Biorad S1000 Thermal Cycler. The thermal cycle was programmed for 30 seconds at 98° C. for initial denaturation, followed by 34 cycles of 10 seconds at 98° C. for denaturation, 30 seconds at 59° C. for annealing, 30 seconds at 72° C. for extension, and 5 minutes at 72° C. for final extension. Human mixed genomic DNA (Promega, Madison, Wis.) and no template were used as negative controls. PCR products were purified using Nucleospin Gel and PCR cleanup as per the manufacturer's instructions (Macherey-Nagel, Duren, Germany). PCR products were then subjected to Sanger sequencing using the Applied Biosystems 3730x1 DNA Analyzer as per manufacturer's instructions (Thermo Fisher, Waltham, Mass.). Output was compared to original candidate fusion sequence and confirmed.

### Droplet Digital PCR

The translocation-primers were designed on both sides of the translocation. One of these primers was used as a common primer for both the translocation and the control. A third primer was designed to be used in combination with the common primer to amplify the wild-type sequence of one of the two translocation partners. The hydrolysis probes labeled with the FAM-fluorochrome at the 5′-end were designed to bind specifically to the translocation PCR-product, while the probes labeled with the HEX-fluorochrome were designed to bind specifically to the control PCR-product. As quenchers, a ZEN quencher was used as an internal quencher, while the Iowa Black FQ-quencher was added to the 3′-end of the probes. Probes were designed to have a higher melting temperature than the primers. The primers and hydrolysis probes were purchased from IDT (Coralville, Iowa, USA). The primers were purified by desalting, while the hydrolysis probes were purified using high-performance liquid chromatography. Upon arrival, primers and probes were resuspended to 100 μM in IDTE (10 mM Tris, pH 8.0; 0.1 mM EDTA) buffer and stored at −20° C. 20 μL droplet digital PCR (ddPCR) reactions were prepared, using 10 μL of 2× ddPCR SuperMix for Probes (No dUTP) (Bio-Rad, Hercules, Calif., USA), 5-30ng of gDNA, as quantified by the Qubit dsDNA high sensitivity assay kit (Thermo Fisher Scientific, Waltham, Mass., USA), primers (each at a final concentration of 900 nM), probes (each at a final concentration of 250 nM) and nuclease-free water. Human mixed genomic DNA (Promega) was used as negative control. Droplets were generated using the QX200 droplet generator (Bio-Rad) by loading the DG8 cartridge (Bio-Rad) with 20 μL of the reaction mixture and 70 μL of droplet generation oil for probes (Bio-Rad). 40 μL of droplet/oil mixture was transferred to a ddPCR 96-well plate (Bio-Rad). The plate was heat-sealed with a pierceable foil heat seal (Bio-Rad). A S1000 Thermal Cycler (Bio-Rad) was used with the following amplification protocol: enzyme activation at 95° C. for 10 minutes, followed by 6 cycles: denaturation at 54° C. for 30 seconds; annealing/extension at 60° C. for 1 minute, followed by 34 cycles: denaturation at 58° C. for 30 seconds; annealing/extension at 60° C. for 1 minute. Following cycling, the samples were held at 98° C. for 10 minutes. Upon completion of the PCR protocol, the plate was read using the QX200 droplet reader (Bio-Rad). Droplet counts and amplitudes were analyzed with QuantaSoft software (v1.7)(Bio-Rad).

### Alignment and Identification of Sequence Alterations

Prior to mutation calling, primary processing of sequence data for samples was performed using Illumina CASAVA software (v1.8.2), including masking of adapter sequences. Sequence reads were aligned against the hg19 human reference genome using ELAND. Candidate somatic mutations in the exome, consisting of point mutations, insertions, and deletions were identified using VariantDx (Jones et al., 2015). To detect mutations that were more likely to be somatic, mutations were excluded that appeared in >10% of the distinct reads and mutations tagged as COMMON or MULT in dbSNP VCF files. Additionally, mutations without a record in COSMIC were excluded as well as in-frame deletions (COSMIC v72). Exceptions to the COSMIC requirement were mutations that predicted truncations in relevant pathways or tumor suppressor genes (Table 5). Single nucleotide polymorphisms (SNPs) flagged as clinically associated or reported in more than 25 samples in COSMIC were not excluded regardless of heterozygosity or percentage of distinct reads. All candidate somatic mutations were confirmed by visual inspection. Samples with more than 2000 alterations after dbSNP filtering were considered hypermutators. Mutational signatures were based on the fraction of mutations in each of the 96 trinucleotide contexts (see, e.g., Alexandrov et al., 2013 Nature 500: 415-421). The contribution of each signature to each tumor sample was estimated using the deconstructSigs R package (Table 14 for R package versions).

### Implementation of DELLY and LUMPY

Identifying probable somatic structural variants in tumor-only experimental designs is a major challenge. False positives arise from germline variants incorrectly reported as somatic and spurious alignments misinterpreted as biological variation. We considered two established tools, DELLY and LUMPY, for detection of structural variants (Rausch et al., 2012; Layer et al., 2014). Reads were aligned to the hg19 reference genome using BWA-MEM (version 0.7.10) (Li and Durbin, 2009) as recommended by these methods. DELLY (version 0.7.7) and LUMPY (version 0.2.13) were implemented using default parameters.

A simple leave-one out cross validation experiment was implemented using the 10 lymphoblastoid controls to evaluate the specificity of these methods for identifying somatic structural variants in a tumor-only experimental design. Specifically, the held out sample was treated as a tumor and identified germline structural alterations in the training set. Excluding structural variants identified in the training set, any alteration identified in the held out sample was considered as a false positive.

### Implementation of Trellis

Germline filters: Using 10 lymphoblastoid cell lines and 8 normal ovarian samples, sequence and germline filters were developed for the hg19 reference genome to flag regions prone to alignment artifacts and/or germline structural variation. Sequence filters for the hg19 reference genome that were masked prior to copy number analyses comprised 326.4 Mb of the genome and included non-overlapping 1 kb genomic intervals (bins) with average mappability less than 0.75 or GC percentage less than 10%, as well as the gaps track from the UCSC genome browser that includes heterochromatin, centromeric, and subtelomeric regions (see, e.g., Fujita et al., 2011 Nucleic Acids Res 39:D876-D882). After removing these sequence filters as well as chrY (all cell lines were derived from women), the read depth was normalized for the remaining 2,680,222 bins. For each bin, the GC-adjusted, log2-transformed count of aligned reads was computed. GC-normalization was implemented using a loess smoother with span ⅓ fitted to a scatterplot of the bin-level GC and log2 count. The GC-adjusted log2 ratios (the residuals from the loess correction) were denoted by R, the mean R for a genomic region by \dot{R̅}, and the median absolute deviation of the autosomal Rs by S. Because some bins had an unusually high or low number of aligned reads in multiple controls, bin i was defined in normal control j as an outlier if |Ri|>(3×Sj). Bins identified as an outlier in two or more normal controls were flagged. These analyses flagged 55,764 genomic regions totaling 75.9 Mb of sequence. To identify somatic copy number alterations, the Rs was segmented using circular binary segmentation implemented in the R package DNAcopy with settings alpha=0.001, undo.splits=‘sdundo’, and undo.SD=2 (see, e.g., Olshen et al., 2004 Biostatistics 5:557-72; and Venkatraman and Olshen, 2007 Bioinformatics 23:657-663). To exclude regions that were either copy number altered in the lymphoblastoid cell lines as well as segments that span difficult regions to genotype, segments having |R|>1 were flagged. A total of 919 segments (46.8 Mb) were flagged across the 18 normal controls.

To characterize copy neutral rearrangements including inversions and translocations in the normal controls, all read pairs were extracted from the BAM file that were improperly paired and for which the intra-mate distance between paired reads was at least 10 kb. A cluster of improper read pairs was defined as a genomic region where at least one base is spanned by five or more improper reads and for which the union of the aligned regions is at least 115 basepairs. Next, these clusters were linked by the mates of the constituent reads. Clusters that could not be linked by at least 5 read pairs were excluded from further analysis. For all linked clusters, at least 90% of the linking read pairs were required to support the same structural variant group (Table 12). Linked clusters for which the type of rearrangement was not consistent among the linking read pairs were excluded from further analysis. For the remaining linked clusters, all the reads supporting the link were realigned using the local aligner BLAT (see, e.g., Kent, 2002 Genome Res 12:656-664). A command-line version of BLAT was utilized for this step (Standalone BLAT v. 35). Confirmation by BLAT required that the reads only align to one location with a BLAT score >90% in the hg19 reference genome. These germline rearrangements were used to screen candidate somatic rearrangements as described in greater detail below.

Somatic deletions: Putative focal homozygous and hemizygous deletions greater than 2 kb and less than 3 Mb in the ovarian cell lines were identified by \dot{R̅}<−3 and \dot{R̅}ϵ (−3; −0:75), respectively. Any deletion ≥75% of the interval were flagged in the control samples were excluded. For each deletion, it was investigated whether any improperly paired reads were aligned within 5 kb of the segmentation boundaries. When five or more rearranged read pairs were aligned near the segmentation boundaries, the distribution of the improper read pair alignments was used to further resolve the genomic coordinates of the deletion boundaries. Resolution of the deletion breakpoints using this approach depends on the intra-mate distance of the improperly paired reads. On average, the intra-mate distance in the ovarian tumors was 262 bp (5th and 95th percentiles: 183 and 353). With multiple rearranged read pairs, it was expected that the resolution of the deletion breakpoints was generally less than 100 bp. As previously described, realignment by BLAT was used to confirm that the rearranged read pairs supporting the deletion mapped uniquely and with high fidelity to this region of the genome. Hemizygous and homozygous deletions supported by rearranged read pairs were indicated by hemizygous+or homozygous+, respectively. Any deletion for which the outlier bins or germline CNVs occupied 75% or more of the width were excluded. Hemizygous deletions not supported by rearranged read pairs were also excluded. All deletions were confirmed by visual inspection.

Somatic amplifications: To identify focal amplicons and establish how these amplicons were linked in the tumor genome, a graph was seeded with high copy focal amplicons. Specifically, putative amplifications were identified as segments with R>1:46, or a 2.75-fold increase from the mean ploidy of the cell line, and between 2 kb and 3 Mb in length. Properly paired reads were used to link seed amplicons to adjacent low-copy duplications (segments with R>0:81 or fold-change of 1.75). When five or more links were established, the low copy segments were added as nodes to the graph with an edge indicating the connection between the high- and low-copy amplicons. Similarly, links were established between the low- and high-copy amplicons that were non-adjacent with respect to the reference genome by analysis of improperly paired reads as previously described.

Somatic copy-neutral intra- and inter-chromosomal translocations and inversions: Candidate somatic copy-neutral rearrangements were identified as previously described in the control samples. However, rearrangements in the ovarian tumor cell lines that overlapped any rearrangement identified in the controls samples were excluded. In addition to improperly paired reads, at least 1 split read supporting the rearrangement was required. To identify split read alignments, all read pairs for which only one read in the pair was aligned within 5 kb of the candidate rearrangement were extracted. For all such read pairs, the unmapped mate was re-aligned using BLAT (see, e.g., Kent, 2002 Genome Res 12:656-664). For any BLAT alignment wherein the realigned read aligned to both ends of the candidate sequence junction with a combined score of the two alignments ≥90% constituted a split read (e.g., FIG. 10).

In-frame gene fusions: To report candidate gene fusions, all candidate somatic rearrangements were identified for which both ends of the novel adjacency in the tumor genome was in a coding region of the genome or a promoter of a gene defined as within 5 kb of the transcription start site. Rearrangements in which both ends resided in the same gene were excluded as these may represent alternative isoforms. For each candidate fusion, two possible orientations of the regions joined in the tumor genome were evaluated and for each orientation the full amino acid sequence of both the 5′ and 3′ transcripts were extracted as well as the candidate amino acid sequence that would be created by the fusion. The fusion was considered to be in-frame if the amino acid sequence of the 3′ partner was a subsequence of the reference amino acid sequence.

### Genome-Wide Methylation Analyses

We pre-processed and normalized raw DAT files from the Infinimum MethylationEPIC array using the funnorm function in the R package minfi (see, e.g., Aryee et al. 2014 Bioinformatics 30:1363-1369). Probes on chromosomes X or Y, probes with detection p-value greater than 0.5, or probes overlapping a SNP with dbSNP minor allele frequency greater than 10% were excluded. In order to understand the similarity of ovarian cells lines with human ovarian cancer, the ovarian cells lines were compared with human ovarian cancer samples available from Genomic Data Commons (gdc.cancer.gov/). The Genomic Data Commons contained 533 human methylation profiles of ovarian cancer and eight normal fallopian tissue samples. Methylation of TCGA ovarian cancers was assessed using Infinium HumanMethylation27 BeadChip array (27,578 probes). The number of probes in common between the HumanMethylation27 platforms and the MethylationEPIC platform was 18,016. On the common set of 18,016 probes, overall methylation was quantified in the TCGA samples and the ovarian cell lines as the fraction of CpG sites with β>0:3. To identify differentially methylated CpG sites comparing normal fallopian tissue to TCGA ovarian cancers, probes were selected from the common set of 18,016 that were hyper-methylated in TCGA ovarian cancer (average β>0:4) and unmethylated in normal fallopian tissue (average β<0:2). In addition, probes were also selected that were hypo-methylated in TCGA ovarian cancer (average β<0:1) and hyper-methylated in normal fallopian (average β>0:3).

### Gene Expression Analyses

Pre-processing and normalization of the 44k Agilent microarray for the ovarian cell lines has been described elsewhere and normalized expression data was available for 44 of the 45 tumors (see, e.g., Konecny et al., 2011 Clinical Cancer Research 17:1591-1602). For copy number altered genes with known clinical relevance to cancer, it was assessed whether amplified genes were over-expressed and whether deleted genes were under-expressed. The probability that a gene was over- or underexpressed was estimated by a two-component hierarchical mixture model implemented in the R package CNPBayes and compared to a single-component mixture model assuming no differential expression. A tumor for which the posterior probability of differential expression was greater than 0.5 was called over- or under-expressed.

### Dose Response Models

Bayesian model averaging: models of the form

logCᵢ=γ₁x_{i,1}+ . . . +γₚx_{i,p},+ϵᵢ.

were considered where Ci denotes the logIC₅₀ and x_{i;j} an indicator for the alteration status (0 not altered, 1 altered) of feature j in cell line i. The regression coefficient for feature j is the product of a binary indicator zj and a real number hj. A modified g-prior was used for γ such that γⱼ was zero whenever zⱼ was zero. For the vector of γ's with non-zero z's, a multivariate normal prior was used. The space of the possible 2p models was explored using a Gibbs sampler. The binary features comprising the x's included somatic mutations, somatic structural variants (deletions, amplifications, in-frame fusions), methylation, and under- or over-expression. For the PARP inhibitor, the number of intra-chromosomal rearrangements and the HRD score as potential markers for HRD were additionally considered. For rearrangements, the mean of the square-root transformed frequency across all cell lines was computed and a binary covariate was defined for whether the square-root transformed statistic was greater than the mean. The HRD score was used without transformation for Bayesian model averaging. For the univariate analyses described in the next section, a binary covariate for HRD was defined according to whether the score was larger than the mean. Qualitatively similar inferences were obtained using the continuous HRD score (data not shown). For the inhibitor of the MEK pathway, one of the logIC₅₀ concentrations was missing. For this cell line, we used the posterior mean from the imputation described in greater detail below.

Univariate analysis of selected features: For a given feature, our sampling model for the length-3 vector of inhibitor concentrations inducing 20%, 50%, and 80% cell death is

logC_{i,altered}=μ+δ+ϵ_{i,altered}

for a cell line with an alteration in this feature and

logC_{i,WT}=μ−δ+ϵ_{i,WT}

for a cell line without an alteration. With inhibitor concentrations on the log scale, the residuals are approximately multivariate-normal:

ϵ_{i,j}˜i.i.d. MVN(0, Σ).

Computationally convenient conjugate priors for the unknown parameters in this model are

p(μ, δ, Σ)=p(μ)p(δ)p(Σ),

μ˜MVN(μ₀, Σ₀),

δ˜MVN(δ₀, Ψ₀)), and

Σ⁻¹˜W(ν₀, S₀⁻¹),

For some cell lines, inhibitor concentrations were incomplete. As the logC were highly correlated across cell lines, missing observations were imputed from the observed data using a Gibbs sampler. Inference regarding differences in mean logC, given by the posterior distribution of 2δ, was based on the marginal probability of the observed data integrating over the missing data. 90% highest posterior density (HPD) intervals were reported for the difference in the mean logIC₅₀.

### Data and Software Availability

Sequencing data will be made available upon publication through the European Genome-phenome Archive at ENSEMBL-EBI (accession EGAS00001002998). The R package Trellis for identifying somatic structural variants in tumor-only analyses is available from github (github.com/cancer-genomics/trellis).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

Table S1 Summary of ovarian cancer cell lines analyzed
Table S2 STR analyses of ovarian cell lines
Table S3 Summary of genomic analyses
Table S4 Summary of sequence alterations
Table S5 Tumor suppressor genes evaluated for inactivating mutations
Table S6 Sequence alterations

### Table S7 Amplifications

### Table S8 Deletions

### Table S9 Rearrangements

Table S10 Predicted in-frame coding fusions
Table S11 Pathway inhibitors
Table S12 Rearrangement types identified from improperly paired reads
Table S13 Primers for Sanger sequencing and droplet digital PCR
Table S14 R package versions
Supplemental Tables for Integrated Genomic, Epigenetic, and Expression Analyses of Ovarian Cancer Cell Lines

Lengthy table referenced here
US20210355545A1-20211118-T00001
Please refer to the end of the specification for access instructions.

Lengthy table referenced here
US20210355545A1-20211118-T00002
Please refer to the end of the specification for access instructions.

Lengthy table referenced here
US20210355545A1-20211118-T00003
Please refer to the end of the specification for access instructions.

Lengthy table referenced here
US20210355545A1-20211118-T00004
Please refer to the end of the specification for access instructions.

Lengthy table referenced here
US20210355545A1-20211118-T00005
Please refer to the end of the specification for access instructions.

Lengthy table referenced here
US20210355545A1-20211118-T00006
Please refer to the end of the specification for access instructions.

Lengthy table referenced here
US20210355545A1-20211118-T00007
Please refer to the end of the specification for access instructions.

Lengthy table referenced here
US20210355545A1-20211118-T00008
Please refer to the end of the specification for access instructions.

Lengthy table referenced here
US20210355545A1-20211118-T00009
Please refer to the end of the specification for access instructions.

Lengthy table referenced here
US20210355545A1-20211118-T00010
Please refer to the end of the specification for access instructions.

Lengthy table referenced here
US20210355545A1-20211118-T00011
Please refer to the end of the specification for access instructions.

Lengthy table referenced here
US20210355545A1-20211118-T00012
Please refer to the end of the specification for access instructions.

Lengthy table referenced here
US20210355545A1-20211118-T00013
Please refer to the end of the specification for access instructions.

Lengthy table referenced here
US20210355545A1-20211118-T00014
Please refer to the end of the specification for access instructions.

LENGTHY TABLES
The patent application contains a lengthy table section. A copy of the table is available in electronic form from the USPTO web site (https://seqdata.uspto.gov/?pageRequest=docDetail&DocID=US20210355545A1). An electronic copy of the table will also be available from the USPTO upon request and payment of the fee set forth in 37 CFR 1.19(b)(3).

1. A method for assessing therapeutic benefit of a therapeutic regimen comprising a PARP inhibitor in a subject comprising:
detecting the presence of a MYC amplification in a tumor sample obtained from the subject, and
identifying that the subject will have a predicted therapeutic benefit from the PARP inhibitor when the presence of the MYC amplification is detected in the tumor sample, wherein the therapeutic benefit for the subject is improved relative to the therapeutic benefit of the PARP inhibitor for a reference subject having a corresponding reference tumor sample that does not exhibit the MYC amplification.

2. (canceled)

3. A method for assessing therapeutic benefit of a therapeutic regimen comprising a PARP inhibitor in a subject determined to have a MYC amplification in a tumor sample obtained from the subject comprising:
identifying that the subject will have a predicted therapeutic benefit from the PARP inhibitor when the presence of the MYC amplification is detected in the tumor sample, wherein the therapeutic benefit for the subject is improved relative to the therapeutic benefit of the PARP inhibitor for a reference subject having a corresponding reference tumor sample that does not exhibit the MYC amplification.

4. (canceled)

5. The method of claim 1, wherein the PARP inhibitor is one or more of talazoparib (BMN-673), olaparib (AZD-2281), rucaparib (PF-01367338), niraparib (MK-4827), veliparib (ABT-888), CEP 9722, E7016, BGB-290, iniparib (BSI 201), 3-aminobenzamide, and combinations thereof.

6-11. (canceled)

12. The method of claim 1, wherein the tumor sample is an ovarian tumor sample.

13. The method of claim 1, further comprising administering a therapeutic regimen to the subject.

14. The method of claim 13, wherein the therapeutic regimen is one or more of:
adoptive T cell therapy, radiation therapy, surgery, administration of a chemotherapeutic agent, administration of an immune checkpoint inhibitor, administration of a targeted therapy, administration of a kinase inhibitor, administration of a signal transduction inhibitor, administration of a bispecific antibody, administration of a monoclonal antibody, and combinations thereof.

15. A method of identifying a cancer-associated alteration in a sample obtained from a subject in the absence of a matched normal sample from the subject comprising:
(a) detection of germline changes, artifactual changes, or both, wherein the detected germline changes and detected artifactual changes are identified as not being a cancer-associated alteration;
(b) detecting the presence of focal homozygous deletions, focal homozygous amplifications, or both, wherein the focal homozygous deletions and focal homozygous amplifications are distinguishable from larger structural changes;
(c) associating one or more copy number regions;
(d) detecting homozygous and hemizygous deletions;
(e) detecting rearrangements using a stringent local re-alignment to detect and remove spurious paired read and split alignments; and
(f) identifying in-frame rearrangements.

16. The method of claim 15, wherein the step of detecting germline changes, artifactual changes, or both comprises applying sequence and germline filters to flag regions prone to alignment artifacts, germline structural variations, or both.

17. The method of claim 15, wherein the step of associating one or more copy number regions comprises generating a plurality of amplicons and comparing paired sequences in the amplicons.

18. The method of claim 17, wherein the step of comparing paired sequences in amplicons comprises generating an undirected graph in which amplicons as nodes and in which edges between amplicons are generated by multiple paired sequencing reads aligned genomic locations associated with the amplicons.

19. The method of claim 15, wherein the step of detecting homozygous and hemizygous deletions comprises detecting copy number changes and rearrangements.

20. The method of claim 15, wherein the identified in-frame rearrangements result in gene fusions.

21. A method of detecting the presence of cancer in a subject comprising performing the steps of claim 15, and further comprising detecting methylation status of one or more genetic loci, which genetic loci are associated with the presence of cancer.

22. The method of claim 21, further comprising administering a therapeutic regimen to the subject.

23. The method of claim 22, wherein the therapeutic regimen is one or more of:
adoptive T cell therapy, radiation therapy, surgery, administration of a chemotherapeutic agent, administration of an immune checkpoint inhibitor, administration of a targeted therapy, administration of a kinase inhibitor, administration of a signal transduction inhibitor, administration of a bispecific antibody, administration of a monoclonal antibody, and combinations thereof.

24. The method of claim 15, wherein the sample is a tumor sample.

25. The method of claim 15, wherein the sample is a liquid biopsy sample.
